SEQUENCE LISTING (1 sequence)

SEQ ID NO: 1 - DNA, 265 bp, Homo sapiens
gaatacgctatcgaacccgaactcctacacccattaagtccgatagtaccgcatcctttatcgggagagacgataacagtaggactaaatctcgccactcctacggacgatatgagacccacctccacacttccggggcagaaccttcctattagataagtgatacccgccttaccccctacgataaaaatggacggcccggcacgaggtcgatcctccggccttatagctatatgggccttccccttaaagacgagcccactgg

(19) United States
(12) Patent Application Publication
(10) Pub. No.: US 2003/0223998 A1
(43) Pub. Date: Dec. 4, 2003

(54) Targeted immunotherapy of acute lymphoblastic leukemia (ALL)

(76) Inventors: Lawrence S. Lamb JR., Columbia, SC (US); Philip Musk, Baltimore, MD (US)

Correspondence Address: Robert S. Thomas, Keenan Building, Third Floor, 1330 Lady Street, Columbia, SC, 29201, US

(21) Appl. No.: 10/375,676

(22) Filed: Feb. 27, 2003

Related U.S. Application Data
(60) Provisional application No. 60/359,689, filed on Feb. 27, 2002.

(51) Int. Cl. A61K 39/395; C12Q 1/68; G01N 33/574; C07H 21/04; C07K 16/30

(52) U.S. Cl. 424/155.1; 424/93.7; 435/6; 435/7.23; 530/388.8; 536/23.2

(57) ABSTRACT

The present invention provides novel methods and compositions for the treatment of cancer, and in particular for acute lymphoblastic leukemia (ALL). The present invention also relates to the use of γδ+ T cells to identify and respond to a specific antigen expressed by ALL, thereby enabling the targeted treatment of ALL. Diagnostic methods and kits for the detection and monitoring of cancer are also provided.

### CROSS-REFERENCE TO RELATED APPLICATIONS

The present application claims the benefit of U.S. Provisional Application Serial No. 60/359,689 filed Feb. 27, 2002, which is incorporated herein by reference thereto.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of grant no. R21 CA 7666701 awarded by the U.S. National Institutes of Health of the Department of Health and Human Services.

### BACKGROUND OF THE INVENTION

1. Field of Invention

The present invention relates to treatments for refractory acute lymphoblastic leukemia (ALL), and in particular, the use of γδ+ T cells to identify and respond to a specific antigen expressed by ALL, thereby enabling the targeted treatment of refractory ALL.

2. Description of Related Art

Cancers develop from uncontrolled multiplication of cells. All cancers are life threatening. Even when cancer does not result in death, it is permanently debilitating, not only to the patient, but also to family, friends and co-workers. Too often, however, cancers prove fatal. The personal and public loss from this cluster of diseases, which cause a significant fraction of all premature deaths, is beyond estimation.

Although effective treatment modalities have been developed in a few cases, many cancers remain refractory to currently available therapies. Refractory acute lymphoblastic leukemia is one such cancer.

Leukemia is a malignant condition of white blood cells in which bone marrow is diffusely replaced by relatively immature white blood cells which generally also appear, in large numbers, in the circulating blood. See Robbins and Angell, Basic Pathology, Second Edition, W. B. Saunders Co., Philadelphia, 349-354 (1976). Leukemias may be classified as acute lymphocytic (or lymphoblastic), chronic lymphocytic, acute myelogenous, or chronic myelogenous.

Acute lymphocytic (or lymphoblastic) leukemia accounts for about 20 percent of all leukemias, occurs predominantly in children, and develops more frequently in males than in females. Untreated, the prognosis for survival is approximately four months; with treatment, survival may be for several years and some cures have been reported (Robbins and Angell, supra).

Although ALL is successfully treated in most cases, resistant forms still pose a significant mortality risk. Acute lymphoblastic leukemia (ALL) is diagnosed in 3000-4000 patients per year, the majority of whom are children. See Cortes, J. E. and Kantarjian, H. M., Cancer 108: 2393-2417 (1995) and Pui, C. H., et al., NEJM 339: 605-615 (1998). The current rate of cure in children varies from 60-80% and is largely due to the development of combination chemotherapy, central nervous system treatment, and newer, intensive therapy for patients at high risk for relapse. Therapy for adult ALL has been less successful, with a cure rate of only 30-40%. See Cortes J. E., et al., Cancer 76: 2393-2417 (1995). Risk-associated factors include high white blood count (WBC), certain cytogenetic abnormalities such as Ph+ ALL or t(4:11) and t(1:19), and a slow response to induction chemotherapy. Although recent advances in the classification of disease and clinical management hold promise for the future, resistant forms of the disease still represent a significant challenge.

The treatment of high-risk ALL in first remission or relapsed ALL has included both dose-intensification of chemotherapy and/or allogeneic bone marrow transplantation (BMT). BMT has the advantage of allowing myeloablative chemotherapy and radiotherapy to reduce the disease burden to minimal levels followed by replacement of hematopoiesis and a potential “graft-versus-leukemia” (GvL) effect based on recognition and subsequent killing of residual ALL by allogeneic immune effector cells. With the possible exception of Ph+ ALL. See Cornelissen, J. J., et al., Blood 97: 1572-1577 (2001). Results have largely been disappointing when compared to other leukemias. See Porter, D. L., et al., Blood 95: 1214-1221(2000). No significant benefit is shown in event-free survival for patients treated with BMT even when combined with post-BMT leukocyte infusions. See Cornelissen, J. J., et al., Blood 97: 1572-1577 (2001) and Wheeler, K. A., et al., Blood 96: 2412-2418 (2000).

Successful immunotherapy of refractory ALL will depend on the presence of a leukemia-associated target antigen accessible to cellular and/or pharmacologic therapy. The GvL effect following BMT is most effective against chronic myeloid leukemia (CML) where the phenomenon was first documented. See Hessner, M. J., et al., Blood 86: 3987 (1995). Targets for GvL may include minor and/or major mismatched histocompatibility antigens and/or leukemia-specific antigens. See Barrett, A. J., Ann NY Acad Sci XX: 203 (1996) and Truitt, R. L., et al., Biol of Blood and Marrow Transplantation 1: 61 (1995).

Every allogeneic BMT patient potentially could benefit from the alloreactive response, although the extent of this benefit varies depending on whether the leukemia expresses MHC Class I to a degree that triggers recognition and killing. It is known that patients who suffer from acute and chronic graft-versus-host disease (GvHD) post-BMT often have a reduced rate of leukemic relapse. See Horowitz, M. M., et al., Blood 75: 555 (1990). This is possibly due to more intense alloreactivity against residual host-derived leukemic cells.

T cell recognition of leukemia-associated antigens is thought to be a potentially important means by which immunocompetent cells may recognize and eliminate the minimal amount of residual leukemia that remains following ablative conditioning therapy. It is generally thought that T lymphocytes recognize and eliminate residual disease through both MHC restricted and non-restricted pathways. See Goldman, J. M., et al., Ann Int Med 108: 806 (1988). Indeed, leukemia associated antigens have been demonstrated on ALL, and include KOR-SA3544 associated with Ph+ ALL. See Mari, T., et al., Leukemia 9: 1233-1239 (1995). Indeed, leukemia associated antigens have been demonstrated on ALL, and include KOR-SA3544 associated with Ph+ ALL. See Mari, T., et al., Leukemia 9: 1233-1239 (1995). In addition they are associated with a polymorphic minor histocompatibility antigen HB-1 associated also with Epstein-Barr virus transformed B cells. See Dolstra, H., et al., J Exp Med 189: 301-308 (1999). Stress induced molecules such as heat shock protein HSP27 have been associated with ALL and AML. See Creagh, E. M., et al., Leukemia 14: 1161-1173 (2000). Also, they have been found to be targets for both MHC-dependent and independent cytolysis. See Multhoff, G., et al., Biol. Chem. 379: 295-300 (1998). The isolation, expansion, and reinfusion of ALL-specific T cells has been elusive, however, and successful specific cellular therapy against ALL has not been documented.

γδ+ T cells mediate non-MHC mediated anti-tumor cytotoxicity via recognition of tumor-associated antigens: Up to ten percent of T cells in normal peripheral blood bear the γδ receptor. See Raulet, D. H., Ann. Rev. Immunol. 7: 175 (1989). Recent reports suggest that γδ+ T cells play a substantially different role in the immune system than that of γδ+ T cells. Most γδ+ T-cells usually do not co-express CD4 or CD8, and therefore may develop normally in the absence of MHC class II molecules. See Bigby, M. et al., J. Immunol. 151: 4465 (1993). Positive selection may not be required. Similarly, it is difficult to elicit a response of γδ+ T cells against allogeneic MHC class I or Class II antigens, and when it has been possible to obtain γδ+ T cell clones against peptide antigens, recognition of these peptides is usually not restricted by classical MHC molecules. See Lanier, L., The Immunologist 3: 182 (1995) and Korngold, R., et al., Transpin. Proc. 44: 335 (1987). In addition, γδ+ T cells tend to recognize intact rather than processed polypeptides.

The anti-tumor role for y+T cells was established by Esslin See Esslin, A., et al., J. Nat. Cancer Inst. 83:1564(1991). He noted that in vitro activated peripheral blood γδ+ T cells posses cytolytic activity to selected human tumor cell lines when compared to similarly activated αβ T cells. This reactivity was not MHC restricted, but was dependent on interaction with LFA-1 b/ICAM1 rather than through the γδ receptor. The cells with anti-tumor activity predominantly expressed the Vγ9/Vδ2 T cell receptor, the predominant circulating γδ+ T cell. Proliferative responses of γδ+ T cells, however, were inhibited by monoclonal antibodies (mAbs) to anti-HLA-A, -B, and -C.

These findings suggest that γδ+ T cells activated through the TCR have an advantage in non-MHC restricted cytolysis, which may correlate with an anti-tumor response. It is known that γδ+ T cells recognize to heat shock proteins. See Kaur, I., et al., J. Immunol. 150: 2046 (1993) and Battistini, L., et al., Mol. Med. 1: 554 (1995). Some of these may be expressed by lymphomas. Vδ1+γδ T cells have been raised against CD48 (TCT.1, Blast-1), the nonclassical stress-related MHC antigens MICA and MICB, EBV-transformed B cells, Burkitt lymphoma, and Daudi lymphoma cells. See Chouaib, F., et al., J. Immunol. 147: 2864 (1991); Steinle, A., et al., Proc. Nat. Acad. Sci. USA 95: 12510 (1998); Hacker, G., et al., J. Immunol. 149: 3984 (1992); and Marx, S., et al., J. Immunol. 158: 2842 (1997). Vδ1+γδ T cells from a patient with B-ALL have been shown to be cytotoxic to certain leukemic cell lines as well. See Duval, M., et al., Leukemia 9: 863 (1995).

Therefore, what is needed is an antileukemic role for γδ+ T cells distinct from the classical APC-αβ T cell interaction that may involve direct recognition of leukemia-associated surface antigen(s) that might be exploited as pharmacologic target(s).

### SUMMARY OF THE INVENTION

The present invention relates to treatments for refractory acute lymphoblastic leukemia (ALL), and in particular, the use of γδ+ T cells to identify and respond to a specific antigen expressed by ALL, thereby enabling the targeted treatment of ALL.

The present invention also relates to a novel method for diagnosing a neoplasia disorder in a mammal, wherein said neoplasia disorder produces a ALL-associated cell surface antigen comprising:

a) providing a sample of biological material from said mammal;

b) contacting said biological material with antibodies specific for the antigen;

c) detecting the presence or absence of an immunological reaction product between said antibodies and said ALL-associated cell surface antigen, the presence of an immunological reaction product being indicative of of said neoplasia disorder in said mammal.

The present invention provides a novel isolated antibody, which specifically binds to an epitope of an ALL-associated cell surface antigen.

In another embodiment, the present invention also provides a novel isolated antibody which specifically binds the same antigen as the monoclonal antibody produced by the hybridoma cell line having ATCC Accession No. #, wherein the purified antibody binds an antigen which exists on a B-cell lymphoma cell line having ATCC Accession No. #, wherein the purified antibody also specifically binds an antigen on antigen-stimulated T-cells, but not on non-stimulated T-cells.

Another embodiment of this invention is an acute lymphoblastic leukemia specific γδ T cell receptor and an isolated polynucleotide which encodes for an acute lymphoblastic leukemia specific γδ T cell receptor.

Another embodiment of this invention is a method of determining the prognosis of a cancer disorder in a subject suffering form such a disorder, comprising detecting the expression of a ALL-associated cell surface antigen with an antibody having ATCC Accession No. #, quantifying the expression levels of the ALL-associated cell surface antigen, and correlating the expression levels with survivability statistics in other subjects.

Another embodiment of the present invention is a novel method of preventing and treating neoplasia disorder or a neoplasia-related disorder in a subject that is in need of such prevention and treatment comprising administering to the subject an antibody that is specific for an ALL-associated cell surface antigen in combination with one or more conventional cancer treatment agents.

Still another embodiment of this invention is a novel method of monitoring the treatment of a cancer disorder in a subject suffering from such a disorder, comprising detecting the expression of a ALL-associated cell surface antigen with an antibody having ATCC Accession No #. and correlating the expression over time of the ALL-associated cell surface antigen in the tissue being diagnosed compared with normal tissue.

The present invention also provides a novel method for inducing or enhancing in a subject an immune response, the method comprising:

a) obtaining a composition comprising an ALL-associated cell surface antigen, or immunogenic fragments of such an antigen, the composition further comprising a pharmaceutically acceptable carrier; and

b) administering a physiologically effective amount of said composition to the subject.

The present invention also provides a novel method for identifying antigens that activate γδ+ T cells comprising deriving a cell line that does not express the antigen(s) recognized by γδ+ T cells from a patient with immunoblastic B cell lymphoma/leukemia, and comparing said cell line to gene expression patterns in populations of acute lymphoblastic leukemia cells that do stimulate a γδ+ T cell response.

Yet another embodiment of this invention is a polynucleotide which encodes for an acute lymphoblastic leukemia specific γδ+ T cell receptor.

The present invention also provides a novel method of treating a neoplasia disorder or a neoplasia-related disorder comprising administering to a subject in need of such treatment an effective amount of an antibody that specifically binds to an ALL-associated cell surface antigen.

Still another embodiment of this invention is a method of treating refractory acute lymphoblastic leukemia in a patient comprising: administering to said patient γδ+ T cell-rich composition in a therapeutically effective amount, wherein the γδ+ T cell-rich composition binds to the acute lymphoblastic leukemia, and lyses the acute lymphoblastic leukemia.

Another embodiment of this invention is a method of treating refractory acute lymphoblastic leukemia in a patient comprising: administering acute lymphoblastic leukemia-specific T cells derived from blood of a bone marrow transplant donor to said patient in a therapeutically effective amount.

Another embodiment of this invention is a method of treating refractory acute lymphoblastic leukemia in a patient, comprising: isolating acute lymphoblastic leukemia-specific T cells from a bone marrow transplant donor, expanding said acute lymphoblastic leukemia-specific T cell, and administering said acute lymphoblastic leukemia-specific T cells into said patient.

Another embodiment of this invention is a method of diagnosing refractory acute lymphoblastic leukemia in a patient, comprising obtaining a sample from said patient, and conducting an assay to detect the presence of a γδ+ T cell, wherein the presence of a leukemia-associated surface antigen on said γδ+ T cell indicates refractory acute lymphoblastic leukemia in said patient.

The present invention also provides a pharmaceutical composition comprising activated γδ+ T cell-rich composition in combination with one or more conventional cancer treatment agents, and a pharmaceutically acceptable carrier.

Another embodiment of the present invention is a pharmaceutical composition comprising an antibody that is specific for an ALL-associated cell surface antigen in combination with one or more conventional cancer treatment agents, and a pharmaceutically acceptable carrier.

Another embodiment of this invention is a cell line that expresses B-acute lymphoblastic leukemia antigens CD19, CD10, and HLA-DR.

Another embodiment of this invention is a method of screening for antigens that activate γδ+ T cells comprising: deriving a cell line that does not express the antigen(s) recognized by γδ+ T cells from a patient with immunoblastic B cell lymphoma/leukemia, and comparing said cell line to gene expression patterns in populations of acute lymphoblastic leukemia cells that do stimulate a γδ+ T cell response.

Another embodiment of this invention is a method of decreasing relapse rates of acute lymphoblastic leukemia post-bone-marrow-transplant in a patient, comprising: administering to said patient a composition having an increased γδ+ T cell level as compared to normal levels for said patient.

Another embodiment of this invention is a method of identifying a compound which is immunogenic to γδ+ T cells comprising: obtaining a culture of activated γδ+ T cells, binding said γδ+ T cells to specific targets, and assaying the immunogenicity of said compounds against said γδ+ T cells.

Another embodiment of this invention is a method for the therapy or prophylaxis of acute lymphoblastic leukemia, which comprises administering to a host in need of the therapy or prophylaxis an effective amount of a γδ+ T cell-rich composition.

Another embodiment of this invention is a pharmaceutical composition comprising: an antibody to a γδ+ T cell antigen that is triggered by the presence of acute lymphoblastic leukemia, and a pharmaceutically acceptable carrier or salt.

Another embodiment of this invention is a method of treatment for acute lymphoblastic leukemia comprising administering to a host in need of the treatment an effective amount of the pharmaceutical composition above.

Yet another embodiment of the present invention is a novel kit for preventing and treating neoplasia disorder or a neoplasia-related disorder in a subject that is in need of such prevention and treatment comprising administering to the subject either activated γδ+ T cell composition or an antibody that is specific for an ALL-associated cell surface antigen in combination with one or more conventional cancer treatment agents.

Another embodiment of this invention is a method of treatment for acute lymphoblastic leukemia comprising administering to a host in need of the treatment an effective amount of the pharmaceutical composition above in combination with an additional therapy selected from the group consisting of chemotherapy, radiation therapy, immunotherapy and toxin therapy.

The present invention also provides a novel method of identifying a compound which is immunogenic to γδ+ T cells comprising:

a) obtaining a culture of activated γδ+ T cells;

b) binding said γδ+ T cells to specific targets, and

c) assaying the immunogenicity of said compounds against said γδ+ T cells.

The present invention also provides a novel pharmaceutical composition comprising an isolated antibody which specifically binds to an epitope of an ALL-associated cell surface antigen, and a pharmaceutically acceptable carrier.

### BRIEF DESCRIPTION OF THE DRAWINGS

FIG. 1A is a chart that shows disease-free survival rates post bone-marrow-transplant (BMT).

FIG. 1B is a chart that shows incidence of relapse from patients with increased γδ+ T cells post-BMT and patients with normal recovery of γδ T cells.

FIG. 2A is a photograph that shows the γδ+ T cells growing in clusters on the ALL.

FIG. 2B is a photograph that shows the γδ+ T cells (after removal and exposure to fresh primary ALL blasts) binding the ALL.

FIG. 2C is a line chart that shows the percentage lysis of the ALL upon exposure of γδ+ T cells.

FIG. 3A is a line chart that shows the cytotoxic activity of in vitro expanded/activated γδ+ T cells from two BMT donors cultured on B cell ALL from the respective recipients+TCR-δ1 monoclonal antibody against lymphoid cell lines.

FIG. 3B is a line chart that shows the cytotoxic activity of in vitro expanded/activated γδ+ T cells from two BMT donors cultured on B cell ALL from the respective recipients+TCR-δ1 monoclonal antibody against myeloid cell lines.

FIG. 4 is a polynucleotide sequence that shows the preliminary Vδ1 chain sequence from the γδ T cell receptor from patients and controls. The underlined region represents an internal C region oligonucleotide probe.

FIG. 5 is a flow cytometry graph gating on the total lymphocyte population from a CD45/SSC plot.

FIG. 6A is a gel photograph of a SDS-PAGE analysis, stained with Coomassie Brilliant Blue, showing mouse soluble T cell receptors (TCRs) purified as described; 4 μg/lane were loaded. 1:6.3, 5:1, and 6:1 denote the Vδ1/Vδ6.3, Vδ5/Vδ1, and Vδ6/Vδ1 TCRs, respectively; a γδ control TCR is also shown. Sizes of marked bands before and after reduction match those predicted for each of the TCRs based on length and N-glycosylation sites.

FIG. 6B is a graph showing that purified soluble TCRs bind to specific anti-TCR mAbs requiring native structure, in a competition assay.

### DETAILED DESCRIPTION OF THE INVENTION

In accordance with the present invention, it has been discovered that γδ+ T cells are activated in vivo and in vitro against primary acute lymphoblastic leukemia (ALL) by direct recognition between the γδ T cell receptor and an ALL-associated cell surface antigen. Thus, the activated γδ+ T cells will bind and lyse primary ALL through the binding of the γδ T cell receptor to an ALL-associated cell surface antigen, which has been identified for the first time herein. Although other cancer cell surface antigens have been reported, never before has an ALL-associated cell surface antigen that is specifically recognized and bound by the γδ T cell receptor been reported. The direct recognition of this ALL-associated cell surface antigen on ALL blasts by the γδ T cell receptor provides many useful therapeutic and diagnostic methods and compositions for the treatment and diagnosis of many neoplasia-related disorders.

The discovery that the γδ T cell receptor recognizes and specifically binds an antigen that is associated with the ALL cell membrane indicates a method of treating a tumor expressing the antigen in a subject comprising injecting into the subject a tumor-inhibiting reagent reactive with the antigen associated with the ALL cell membrane. The reagent can be an antibody or an antibody attached to a cytotoxic or cytostatic agent. The cytotoxic or cytostatic agent can, for example, be selected from the group consisting of toxins, radiolabeled moieties, and chemotherapeutic agent. The invention further provides a method of detecting the antigen on tumor cells such as obtained from a biopsy comprising contacting the tumor cells with a reagent specifically reactive therewith and detecting the bound reagent.

Therefore, the antibodies, including monoclonal antibodies, that are directed to this ALL-associated cell surface antigen, provide an unexpectedly effective therapy for neoplasia. Such antibody compositions have a wide range of antitumor and anticancer activity as well as the inhibition of metastasis. This treatment has less systemic toxicity than a conventional chemotherapy treatment alone.

These antibodies also provide diagnostic methods and kits for the detection, prognosis and treatment monitoring of several neoplasia disorders, including such leukemia-type neoplasia as ALL. Other possible uses include the detection of minimal residual disease, patient evaluation for specific cellular or antibody therapy, and evaluation of patient response to treatment.

It has also been discovered that the treatment or prevention of neoplasia and neoplasia-related disorders, used interchangeable herein, including such neoplasia disorders as leukemia, colorectal cancer, lung cancer, and breast cancer, is provided by administering as a monotherapy the novel antibody compositions described herein to a subject suffering from or needing prevention of a neoplasia or neoplasia-related disorder.

As used herein, the term “activated”, when used to describe γδ+ T cells, means the point in time at which the γδ+ T cells have recognized the ALL-associated cell surface antigen.

As used herein, the term “allogeneic” means involving, derived from, or being individuals of the same species that are sufficiently unlike genetically to interact antigenically.

As used herein, the term “ALL” refers to acute lymphoblastic leukemia.

As used herein, the terms “ALL-associated cell surface antigen”, means the antigen found on, or substantially on, the surface of ALL blasts that is specifically recognized by the γδ T cell receptor having a polynucleotide sequence comprising the partial polynucleotide sequence of SEQ ID NO. 1. The present invention encompasses situations where the antigen is only partially expressed on the surface of the ALL blasts, while the rest is, for example, partially expressed beneath the ALL cell surface as a trans-membrane protein.

As used herein, the term “assay” refers to a procedure that is conducted to determine the amount of a particular constituent of a mixture or of the biological or pharmacological potency of a drug.

As used herein, the term “autologous” means derived from the same individual.

As used herein, the term “binding” refers to a non-covalent or a covalent interaction, preferably non-covalent, that holds two molecules together. For example, two such molecules could be an enzyme and an inhibitor of that enzyme. Non-covalent interactions include hydrogen bonding, ionic interactions among charged groups, van der Waals interactions and hydrophobic interactions among nonpolar groups. One or more of these interactions can mediate the binding of two molecules to each other.

As used herein, the term “BMT” refers to bone marrow transplant.

As used herein, the term “CD4” refers to 55-kd glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

As used herein, the term “CD8” refers to differentiation antigens found on thymocytes and on cytotoxic and suppressor T-lymphocytes. Cd8 antigens are members of the immunoglobulin supergene family and are associative recognition elements in MHC (major histocompatibility complex) class I-restricted interactions.

As used herein, the term “CD10” refers to a common acute lymphoblastic leukemia antigen which is expressed by T-cells and B-cells during early stages of development.

As used herein, the term “CD19” refers to differentiation antigens expressed on B-lymphocytes and B-cell precursors. They are involved in regulation of B-cell proliferation.

As used herein, the term “cDNA” means complementary deoxyribonucleic acid. “Complementary” polynucleotides are those which are capable of base-pairing according to the standard Watson-Crick complementarity rules. Specifically, purines will base pair with pyrimidines to form combinations of guanine paired with cytosine (G:C) and adenine paired with either thymine (A:T) in the case of DNA, or adenine paired with uracil (A:U) in the case of RNA. Two polynucleotides may hybridize to each other if they are complementary to each other, or if each has at least one region that is substantially complementary to the other.

As used herein, the term “DNA” means deoxyribonucleic acid.

As used herein, the term “ELISA” means enzyme-linked immunosorbent assay.

As used herein, the term “effective amount” refers to the amount required to produce the desired effect.

As used herein, the term “gene” should be understood to refer to a unit of heredity. Each gene is composed of a linear chain of deoxyribonucleotides which can be referred to by the sequence of nucleotides forming the chain. Thus, “sequence” is used to indicate both the ordered listing of the nucleotides which form the chain, and the chain, itself, which has that sequence of nucleotides. (“Sequence” is used in the similar way in referring to RNA chains, linear chains made of ribonucleotides). The gene may include regulatory and control sequences, sequences, which can be transcribed into an RNA molecule, and may contain sequences with unknown function. The majority of the RNA transcription products are messenger RNAs (mRNAs), which include sequences which are translated into polypeptides and may include sequences which are not translated.

As used herein, the term “high stringency hybridization conditions” refers to hybridization in 50% formamide, 1 M NaCL, 1% SDS at 37° C., and a final wash in 0.1×SSC at 60° C. Methods for nucleic acid hybridizations are described in Meinkoth and Wahl; Ausubel et al.; and Tijssen. See Meinkoth et al., Anal Biochem 138: 267-284 (1984); Current Protocols in Molecular Biology, Chapter 2, Ausubel et al. Eds., Greene Publishing and Wiley-Interscience, New York (1995) and Laboratory Techniques in Biochemistry and Molecular Biology: Hybridization with Nucleic Acid Probes, Part I, Chapter 2, Tijssen, Elsevier, New York (1993).

As used herein, the term “HLA-DR” refers to an antibody bound to an MHC-II molecule.

As used herein, the term “JM1 cell line” refers to a cell line derived from lymphoblastic lymphoma which does not appear to stimulate γδ T cells but has similar properties to acute lymphoblastic leukemia.

As used herein, the term “mRNA” means messenger ribonucleic acid.

As used herein, “nucleic acid” and “polynucleotide” refers to RNA or DNA that is linear or branched, single or double stranded, or a hybrid thereof. The term also encompasses RNA/DNA hybrids. Less common bases, such as inosine, 5-methylcytosine, 6-methyladenine, hypoxanthine and others can also be used for antisense, dsRNA and ribozyme pairing. For example, polynucleotides which contain C-5 propyne analogues of uridine and cytidine have been shown to bind RNA with high affinity and to be potent antisense inhibitors of gene expression. Other modifications, such as modifications to the phosphodiester backbone, or the 2′-hydroxy in the ribose sugar group of the RNA can also be made. The antisense polynucleotides and ribozymes can consist entirely of ribonucleotides, or can contain mixed ribonucleotides deoxyribonucleotides. The polynucleotides of the invention may be produced by any means, including genomic preparations, cDNA preparations, in vitro synthesis, RT-PCR and in vitro or in vivo transcription.

As used herein, the terms “percent sequence identity” between two polynucleotide or two polypeptide sequences is determined according to the either the BLAST program (Basic Local Alignment Search Tool; Altschul and Gish (1996) Meth Enzymol 266:460-480 and Altschul (1990) J Mol Biol 215:403-410) in the Wisconsin Genetics Software Package (Devererreux et al. (1984) Nucl Acid Res 12:387), Genetics Computer Group (GCG), Madison, Wis. (NCBI, Version 2.0.11, default settings) or using Smith Waterman Alignment (Smith and Waterman (1981) Adv Appl Math 2:482) as incorporated into GeneMatcher Plus™ (Paracel, Inc., http://www.paracel.com/html/genematcher.html; using the default settings and the version current at the time of filing). It is understood that for the purposes of determining sequence identity when comparing a DNA sequence to an RNA sequence, a thymine nucleotide is equivalent to a uracil nucleotide.

As used herein, the term “polypeptide” is meant a chain of at least two amino acids joined by peptide bonds. The chain may be linear, branched, circular or combinations thereof. Preferably, polypeptides are from about 10 to about 1000 amino acids in length, more preferably 10-50 amino acids in length. The polypeptides may contain amino acid analogs and other modifications, including, but not limited to glycosylated or phosphorylated residues.

As used herein, the term “prophylaxis” refers to measures designed to preserve health (as of an individual or of society) and prevent the spread of disease.

As used herein, the term “refractory” means not readily yielding to treatment.

As used herein, the term “T cell” refers to a class of lymphocytes, so called because they are derived from the thymus and have been through thymic processing. They are involved primarily in controlling cell-mediated immune reactions and in the control of B cell development. The T cells coordinate the immune system by secreting lymphokine hormones.

As used herein, the term “toxin therapy” refers to therapy using a toxin.

As used herein, the term “treating” or “treatment” as used herein covers any treatment of a disease and/or condition in a mammal, particularly a human, and includes:

(i) preventing a disease, disorder or condition from occurring in a mammal which may be predisposed to the disease, disorder and/or condition but has not yet been diagnosed as having it;

(ii) inhibiting the disease, disorder or condition, i.e., arresting its development; and

(iii) relieving the disease, disorder or condition, i.e., causing regression of the disease, disorder and/or condition.

As used herein, the term “subject” for purposes of treatment includes any subject, and preferably is a subject who is in need of the treatment of neoplasia or a neoplasia-related disorder. For purposes of prevention, the subject is any subject, and preferably is a subject that is at risk for, or is predisposed to, developing neoplasia or a neoplasia-related disorder.

As used herein, the terms “predisposed to” and “at risk for,” both of which are used interchangeably herein, mean any subject at risk for developing neoplasia or at risk for re-developing neoplasia during a relapse of such a disorder. For example, after treatment, many neoplasia disorders subside into remission, meaning that the disease is present, but inactive within the subject and is thus, capable of re-developing at a later time. The subject may be at risk due to genetic predisposition, diet, lifestyle, age, exposure to radiation, exposure to neoplasia-causing agents, and the like.

As used herein, the terms “subject in need of” refer to any subject who is suffering from or is predisposed to neoplasia or any neoplasia-related disorder described herein. The terms “subject in need of” also refer to any subject that requires a lower dose of conventional neoplasia treatment agents. In addition, the terms “subject in need of” means any subject who requires a reduction in the side-effects of a conventional treatment agent. Furthermore, the terms “subject in need of” means any subject who requires improved tolerability to any conventional treatment agent for a neoplasia disorder therapy.

The subject is typically an animal, and yet more typically is a mammal. “Mammal”, as that term is used herein, refers to any animal classified as a mammal, including humans, domestic and farm animals, zoo, sports, or pet animals, such as dogs, horses, cats, cattle, etc. The subject may also be a human subject who is at risk for developing neoplasia or at risk for a relapse of a neoplasia disorder.

The terms “neoplasia” and “neoplasia-related disorder” refers to new cell growth that results as a loss of responsiveness to normal growth controls, e.g. to neoplastic cell growth. Neoplasias include “tumors,” which may be either benign, premalignant or malignant. As used herein, the term “neoplasia” also encompasses other cellular abnormalities, such as hyperplasia, metaplasia and dysplasia.

For purposes of the present invention, the terms “neoplasia”, “metaplasia”, “dysplasia” and “hyperplasia” can be used interchangeably herein, as their context will reveal, referring generally to cells experiencing abnormal cell growth. Hyperplasia is an absolute increase in the number of cells per unit of tissue, is generally initiated and regulated by definable, such as hormonal, stimuli, and may be useful to the host (physiologic and adaptive hyperplasia). Metaplasia denotes a change of one type of adult cell to another, is usually an adaptive response to an inflammatory or other abnormal stimulus, and is often reversible. Dysplasia is an abnormal a typical cellular proliferation (a typical hyperplasia), is usually reversible, and is not a tumor but possibly a precursor in some circumstances. For purposes of the present invention, the use of the term “neoplasia” is intended to encompass metaplasia, dysplasia and hyperplasia.

The present invention relates to methods for inhibiting the growth of neoplasia, including a malignant tumor or cancer comprising exposing the neoplasia to an inhibitory or therapeutically effective amount or concentration of at least one of the disclosed compositions. This method may be used therapeutically, in the treatment of neoplasia, including cancer or in comparison tests such as assays for determining the activities of related analogs as well as for determining the susceptibility of a patient's cancer to one or more of the compounds according to the present invention.

Thus, the present invention encompasses a method of treating a neoplasia disorder or a neoplasia-related disorder comprising administering to a subject in need of such treatment an effective amount of an antibody that specifically binds to an ALL-associated cell surface antigen.

In still another embodiment, the present invention encompasses a method of treating a neoplasia disorder or a neoplasia-related disorder comprising administering to a subject in need of such treatment an effective amount of an antibody that specifically binds to an ALL-associated cell surface antigen, wherein said antibody has a cytotoxic activity.

In yet another embodiment, the present invention encompasses a method of treating a neoplasia disorder or a neoplasia-related disorder comprising administering to a subject in need of such treatment an effective amount of an antibody that specifically binds to an ALL-associated cell surface antigen, wherein said antibody has a cytotoxic activity, wherein said antibody has a cytotoxic activity that is selected from the group consisting of perforin, granzyme A, granzyme B and Fas-mediated.

In other embodiments, the present invention encompasses a method of treating a neoplasia disorder or a neoplasia-related disorder comprising administering to a subject in need of such treatment an effective amount of an antibody that specifically binds to an ALL-associated cell surface antigen, wherein said antibody specifically binds the same ALL-associated cell surface antigen as the monoclonal antibody produced by the hybridoma cell line having ATCC Accession No. # or wherein said ALL-associated cell surface antigen is specifically recognized by a γδ T cell receptor.

The present invention also encompasses a method of treating a neoplasia disorder or a neoplasia-related disorder comprising administering to a subject in need of such treatment an effective amount of an antibody that specifically binds to an ALL-associated cell surface antigen, wherein the γδ T cell receptor comprises a polynucleotide sequence having at least 70% to 100% of the sequence identity to SEQ ID NO.1.

In other embodiments, the neoplasia disorder is a leukemia disorder. In still other embodiments, the leukemia disorder is a lymphocytic leukemia disorder or a myelogenous leukemia disorder. In yet other embodiments, the neoplasia disorder is selected from the group consisting of acute myelogenous leukemia, myeloblastic leukemia, promyelocytic leukemia, myelomonocytic leukemia, monocytic leukemia, erythroleukemia, megakaryoblastic leukemia, chronic myelogenous leukemia, acute lymphocytic leukemia, chronic lymphocytic leukemia, and hairy cell leukemia.

The present invention also encompasses a method for inducing or enhancing in a subject an immune response, the method comprising:

a) obtaining a composition comprising an ALL-associated cell surface antigen, or immunogenic fragments of such an antigen, the composition further comprising a pharmaceutically acceptable carrier; and

b) administering a physiologically effective amount of said composition to the subject.

In other embodiments, the present invention encompasses a method of determining the prognosis of a cancer disorder in a subject suffering form such a disorder, comprising detecting the expression of a ALL-associated cell surface antigen with an antibody having ATCC Accession No. #, quantifying the expression levels of the ALL-associated cell surface antigen, and correlating the expression levels with survivability statistics in other subjects.

In still other embodiments, the present invention encompasses a method of monitoring the treatment of a cancer disorder in a subject suffering from such a disorder, comprising detecting the expression of a ALL-associated cell surface antigen with an antibody having ATCC Accession No. #, and correlating the expression over time of the ALL-associated cell surface antigen in the tissue being diagnosed compared with normal tissue.

In yet other embodiments, the present invention encompasses a method for identifying antigens that activate γδ+ T cells comprising deriving a cell line that does not express the antigen(s) recognized by γδ+ T cells from a patient with immunoblastic B cell lymphoma/leukemia, and comparing said cell line to gene expression patterns in populations of acute lymphoblastic leukemia cells that do stimulate a γδ+ T cell response.

In other embodiments, the present invention also encompasses a novel kit for preventing and treating neoplasia disorder or a neoplasia-related disorder in a subject that is in need of such prevention and treatment comprising administering to the subject an antibody that is specific for an ALL-associated cell surface antigen in combination with one or more conventional cancer treatment agents.

In still further embodiments, the present invention encompasses a novel kit for preventing and treating neoplasia disorder or a neoplasia-related disorder in a subject that is in need of such prevention and treatment comprising administering to the subject an activated γδ+ T cell-rich composition in combination with one or more conventional cancer treatment agents.

In other embodiments, the present invention encompasses a cell line that expresses B-acute lymphoblastic leukemia antigens CD19, CD10, and HLA-DR.

In other embodiments, the present invention encompasses a method of identifying a compound which is immunogenic to γδ+ T cells comprising:

a) obtaining a culture of activated γδ T cells;

b) binding said γδ+ T cells to specific targets, and

c) assaying the immunogenicity of said compounds against said γδ+ T cells.

In other embodiments, the present invention encompasses a method for detecting a cancer disorder in a subject comprising contacting an antibody which specifically binds the same ALL-associated cell surface antigen as the monoclonal antibody produced by the hybridoma cell line having ATCC Accession No. #, with a biological tissue or fluid sample obtained from said subject and detecting interaction of said antibody with any antigenically corresponding tumor cells or antigenic determinants thereof in said sample by observing a detectable signal produced by the interaction of said antibody with said tumor cells or antigenic determinants thereof.

In other embodiments, the present invention encompasses a monoclonal antibody produced by the hybridoma cell line deposited with the ATCC as accession No # and as well the cell line deposited with the ATCC as accession No #.

The present invention also encompasses the development of novel methods for the future screening of other immunologically potent compounds using the ALL-associated cell surface antigen described herein.

The present invention also provides a novel antibody composition that has been discovered to be a potent neoplasia treatment therapy, alone and in combination with conventional treatment agents.

Also provided is a neoplasia treatment therapy comprising the novel antibody composition described herein in combination with the administration of activated γδ+ T cells to a subject in need of such therapy. The administration of the antibody compositions described herein is unexpectedly an effective treatment therapy for the prevention and treatment of neoplasia. Such administration is effective for preventing and treating the symptoms of neoplasia without the disadvantages and side effects associated with current treatment strategies. For example, conventional chemotherapy treatments are known to have many undesirable side effects. Also, surgical and radiotherapy treatments are often infective for treating newly formed and relatively small neoplasms.

The present invention also provides a method for lowering the required dosages of conventional cancer treatment agents for neoplasia therapy. Also, the present invention provides a method for increasing the effectiveness of conventional cancer treatment therapies, including chemotherapy, radiation therapy and surgical intervention.

For example, the administration of activated γδ+ T cells in combination with one or more conventional cancer treatment agents to a subject in need of the prevention or treatment of neoplasia is unexpectedly superior to the use of either agent alone. The combination therapy of the present invention is thus, effective for lowering the dosages of conventional chemotherapy and radiation therapy treatments that are normally prescribed as a monotherapy. The administration of lower dosages of conventional treatment agents provides a reduction in side effects corresponding to such conventional agents.

Moreover, in one embodiment, the combination therapy demonstrates a synergistic efficacy for treating and preventing neoplasia that is greater than what would be expected from simply combining the two therapies. By use of the term “synergistic”, it is meant that any of the individual treatment methods and compositions described herein and administered to a subject alone provides a given and expected level of efficacy. However, a synergistic effect is one in which the combined treatment methods give a level of efficacy that is greater than what would be expected from simply the sum of the individual treatments.

However, in other embodiments, it is not necessary that the combination treatment and prevention therapies render a synergistic result. For example, the combination may useful without the need for being synergistic such as for increasing shelf-life or reducing toxicity.

As used herein, the term “therapeutically effective” is intended to qualify the amount of an agent for use in therapy, which will achieve the goal of preventing, or improvement in the severity of, the disorder being treated, while avoiding adverse side effects typically associated with alternative therapies. A neoplasia symptom is considered ameliorated or improved if any benefit is achieved, no matter how slight. For example, any detectable reduction in the size of tumor in a subject would be considered an ameliorated symptom. Thus, an amount of an antibody that is specific for the ALL-associated cell surface antigen or an amount of either activated γδ+ T cells or conventional cancer treatment agents that causes a decrease in the frequency of incidence is “prophylactically effective”, where the term “prophylactic” refers to the prevention of disease, whereas the term “therapeutic” refers to the effective treatment of existing disease.

As used herein, the phrases “combination therapy”, “co-administration”, “co-administering”, “administration with”, “administering”, “combination”, or “co-therapy”, when referring to the use of an antibody that is specific for the ALL-associated cell surface antigen or the use of activated γδ+ T cells or conventional cancer treatment agents, are intended to embrace administration of each agent in a sequential manner in a regimen that will provide beneficial effects of the drug combination, and is intended as well to embrace co-administration of these agents in a substantially simultaneous manner. Thus, combinations of the antibody composition or activated γδ+ T cells and conventional treatment agents may be administered in one therapeutic dosage form, such as in a single capsule, tablet, or injection, or in two or three separate therapeutic dosage forms, such as in separate capsules, tablets, or injections.

Sequential administration of such treatments encompasses both relatively short and relatively long periods between the administration of each of the therapies of the present method. However, for purposes of the present invention, the second and third therapies are administered while the first therapy is still having an efficacious effect on the subject. Thus, the present invention takes advantage of the fact that the simultaneous presence of combinations of the antibody composition described herein or activated γδ+ T cells and conventional treatment agents in a subject has a greater efficacy than either one alone.

Preferably, the second or third of the therapies is to be given to the subject within the therapeutic response time of the first therapy to be administered. For example, the present invention encompasses administration of activated γδ+ T cells to the subject and the later administration of one or more conventional cancer treatment agents, as long as the conventional treatment agent is administered to the subject while the activated γδ+ T cells are still present in the subject at a level, which in combination with the level of the conventional treatment agent is therapeutically effective, and vice versa.

The novel composition of the invention may be used in conjunction with other treatment modalities. The present invention may be used in conjunction with any current or future therapy, including, but not limited to radiation, chemotherapy, surgical therapy, immunotherapy and cryotherapy.

Thus, for purposes of the present, the term “conventional treatment agent” or “conventional cancer treatment agent” encompasses radiation therapy, chemotherapy, surgical therapy, immunotherapy and cryotherapy.

Radiation therapy utilizes x-rays, electrons and other types of radiation such as iodine-131 to treat cancer and benign disease. Radiation can be used alone or in conjunction with various treatments to cure cancer and other serious ailments. When a cure is not possible, radiation therapy can often relieve symptoms and provide indispensable relief.

For cancer treatment, radiation therapy is often used before surgery to shrink malignant tumors, after surgery to stop the growth of cancer cells, and in combination with chemotherapy to destroy cancer or prevent its reappearance. Radiation therapy has been more successful with some cancers than with others. It is likely that the combination of radiation with therapies of the present invention in some cases will be synergistic.

Chemotherapy has been more successful with some cancers than with others. It is likely that the combination of chemotherapy with therapies of the present invention in some cases will be synergistic.

“Chemotherapeutic agents”, as used herein, encompass all chemotherapeutic agents or drugs used in the treatment of malignancies. This term is used for simplicity notwithstanding the fact that other compounds may be technically described as chemotherapeutic agents in that they exert an anti-cancer effect.

The antibody or antigen compounds of the present invention may be administered to a subject in need of such therapy in combination with one or more conventional cancer treatment agents, including chemotherapeutic agents. Likewise, the activated γδ+ T cells of the present invention may be administered to a subject in need of such therapy in combination with one or more conventional cancer treatment agents. In other embodiments, the antibody or antigen compounds of the present invention may be administered to a subject in need of such therapy in combination with one or more conventional cancer treatment agents.

In one embodiment, the chemotherapy conventional cancer treatment agents that are suitable for use with the present invention include those agents selected from the group consisting of antimetabolite agents, adjunctive agents, alkylating agents, antibiotic-type agents, antimetastatic agents, immunotherapeutic agents, interferon-type agents, vinca alkaloids, asparaginases, podophylotoxins, devices, vaccines, radiotherapeutic agents, hormonal anticancer agents, nitrosoureas, and mixtures of two or more thereof.

In another embodiment, the conventional cancer treatment agents that are suitable for use with the present invention include those agents selected from the group consisting of antineoplastic agent is selected from the group consisting of anthracyclines, DNA intercalators, anti-cancer antibiotics or antibiotic-type agents, bisphosphonate agents, cGMP phosphodiesterase inhibitors, calcium carbonate, DHA derivatives, DNA topoisomerase inhibitors, epipodophylotoxins, genistein, hydrophilic bile acids (URSO), interferon antagonists, monoclonal antibodies other than those provided by the present invention, ornithine decarboxylase inhibitors, radio/chemo sensitizers/protectors, retinoids, selective inhibitors of proliferation and migration of endothelial cells, selenium, stromelysin inhibitors, synthetic nucleosides, taxanes and taxane derivatives, retinoids, angiotensin-converting enzyme (ACE) inhibitors, analgesic agents, genistein agents, 5-HT 3 antagonists, lipoxygenase inhibitors, acetylcholinesterase inhibitors, ACTH releasing factor, acyltransferase inhibitor, adenosine A3 agonist and antagonists, adenosine deaminase inhibitor, adenosine modulator, adenosylhomocysteinease inhibitor, adenylate cyclase stimulator, alpha 1 adrenoceptor antagonist, alpha 2 adrenoceptor antagonist, alpha glucosidase inhibitor, alpha glycosidase inhibitor, alpha mannosidase inhibitor, alpha reductase inhibitor, aminopeptidase inhibitor, androgen antagonist, antihypercholesterolemic agent, antihyperlipidemic agent, antimicrobial agent, antioxidant agent, apoptosis inhibitor, apoptosis modulator, apoptosis stimulator, arginine modulator, aromatase inhibitor, asparaginase stimulator, aspartate carbamoyltransferase inhibitor, ATPase inhibitor, B cell differentiating factor, bile acid modulator, bioreducible cytotoxin, BK agonist, bombesin antagonist, bone metabolism modulator, bone resorption inhibitor, bradykinin BK-1 antagonist, BZD agonist, calcitonin agonist, calcium channel activator, calcium channel blocker, calcium metabolic inhibitor, cathepsin inhibitor, CCK antagonist, cell adhesion inhibitor, cell adhesion modulator, cell adhesion molecule antagonist, cell control agent, cell cycle inhibitor, cell surface receptor inhibitor, cell wall synthesis inhibitor, chelating agent, chemokine, chemoprotectant, chemosensitizer, chemotactic factor, chloride channel blocker, chorionic gonadotropin, coagulation inhibitor, collagenase inhibitor, complement cascade modulator, CSF 1 agonist, cysteine protease inhibitor, cytokine agonist, cytokine antagonist, cytokine modulator, cytokine release inhibitor, cytokine synthesis modulator, dehydrogenase inhibitor, DHFR inhibitor, dihydropteroate pyrophosphorylase inhibitor, dihyrdropyrimidine dehydrogenase inhibitor, DNA gyrase inhibitor, DNA intercalator, DNA modulator, DNA polymerase inhibitor, DNA RNA polymerase inhibitor, DNA synthesis inhibitor, DNA vaccine, DNase modulator, dopamine D2 agonist, p-glycoprotein inhibitors, EGF antagonist, EGF binding agent, HER-2 antagonist, elastase inhibitor, electron transport inhibitor, endothelial growth factor antagonist, endothelian converting enzyme inhibitor, epidermal growth factor antagonist, estradiol and estradiol derivatives, estradiol 17 beta dehydrogenase stimulator, estradiol agonist, estrogen agonist, estrogen antagonist, FGF agonist, FGF antagonist, folate antagonist, folate synthesis inhibitor, fucosidase alpha modulator, G protein modulator, GAR transformylase inhibitor, gastrin antagonist, GCSF, gelatinase inhibitor, glutamate antagonist, glutathione transferase inhibitor, glutathione transferase stimulator, glycosidase inhibitor, GM-CSF agonist and antagonist, GNRH agonist, GNRH antagonist, growth factor agonist, growth factor antagonist, growth hormone agonist and antagonists, growth hormone releasing factor antagonist, guanylate cyclase inhibitor, H2 agonist, hematopoietic inhibitor, hematopoietic modulator, hematopoietic stimulant, hemoglobin modulator, heparin binding agent, heparin modulator, heparinase inhibitor, hepatocyte growth factor antagonist, histamine modulator, HIV protease inhibitor, HMG CoA reductase inhibitor, hyaluronic acid inhibitor, hydroxylase inhibitor, ICE inhibitor, IFN agonist, IFN alpha, IFN alpha 2, IFN beta, IFN beta agonist, IFNγ, IFNγ agonist, IFN omega, interleukin-1, interleukin-1 agonist, interleukin-1 alpha, interleukin-1 antagonist, interleukin-1 beta, interleukin-1 release inhibitor, interleukin-1 synthesis inhibitor, interleukin-10, interleukin-10 agonist, interleukin-12, interleukin-13, interleukin-2, interleukin-2 agonist, interleukin-2 antagonist, interleukin-2 synthesis modulator, interleukin-3 agonist, interleukin-4, interleukin-4 agonist, interleukin-6, interleukin-6 agonist and antagonist, interleukin-8, interleukin-9, interleukin antagonist interleukin agonist, interleukin synthesis modulator, immunostimulants, immunosuppressant, immunotoxin, IMP dehydrogenase inhibitor, inhibin, inotropic agent, insulin-like growth factor-1, insulin agonist, interferon modulator, ion channel modulator, isomerase inhibitor, keratolytic, leukocyte elastase inhibitor, LHRH, LHRH agonist and antagonist, lyase inhibitor, lysase inhibitor, antibiotics, macrophage migration inhibitory factor, MAO inhibitor, mast cell degranulation inhibitor, megakaryocyte growth factor, melanin, melatonin ligand, methionine synthase inhibitor, microtubule inhibitor, NGF antagonist, NK1 antagonist, NMDA antagonist, NO synthesis inhibitor and modulator, nootropic agent, nucleic acid metabolism modulator, oncogene inhibitor, ornithine decarboxylase inhibitors and stimulators, osteogenesis stimulator, oxidoreductase inhibitor, P450 reductase inhibitor, PABA antagonist, PAF antagonist, PAI inhibitor, phosphodiesterase inhibitor, PDGF antagonist, peptide agonist, permeability enhancer, prostaglandin agonists, MCP-1 inhibitor, MAP kinase inhibitors, phosphokinase inhibitors, phospholipase C inhibitor, photosensitizers, PLA2 inhibitor, plasminogen activator inhibitor, platelet aggregation inhibitor, polyamine oxidase inhibitor, polyamine synthesis inhibitor, progesterone ligand, progestogen agonists and antagonists, prostacyclin agonist, protease inhibitor and modulator, protein farnesyl transferase inhibitor, protein kinase A inhibitor, protein kinase C inhibitor, protein kinase inhibitor, protein synthesis inhibitor, PTH antagonist, purine nucleoside phosphorylase inhibitor, radiochemosensitizer, radioimmuno-therapeutic, radiopharmaceutical, radioprotectant, radiosensitizer, RAS protein inhibitor and modulators, retinoid modulator, retinoid receptor agonist and antagonist, retinoid receptor ligand, reverse transcriptase inhibitor, ribonucleotide reductase inhibitor, ribosomal binding inhibitor, ribosomal metabolic modulator, ribosome binding agent, RNA modulator, RNA polymerase inhibitor, RNA synthesis inhibitor, S adenosylmethionine decarboxylase inhibitor, selectin antagonist, serine protease inhibitor, somatostatin agonist, somatostatin analog, somatostatin modulator, squalene synthetase inhibitor, steroid agonist, steroid reductase inhibitor, sterol demethylase inhibitor, substance P antagonist, telomerase inhibitor and modulator, testosterone 5 alpha reductase inhibitor, testosterone modulator, TGF alpha, TGF beta-2, TGF beta antagonist, thrombin inhibitor, thymidine kinase inhibitor, thymidine kinase modulator, thymidylate synthase inhibitor, thyrotropin, TNF-alpha, TNF agonist, TNF alpha antagonist, TNF alpha synthesis inhibitor, TNF antagonist, TNF modulator, TNF beta, IFN agonist, TNF synthesis stimulator, topoisomerase inhibitor, topoisomerase-I inhibitor, topoisomerase-II inhibitor, transferase inhibitor and stimulator, tubulin agonist, tubulin binding agent, tubulin modulator, TXA2 antagonist, tyrosinase inhibitor, tyrosine kinase inhibitor, urokinase inhibitor, vaccines, viral replication inhibitor, vitamin B12 agonist, vitamin D agonist, vitamin D2 agonist, vitamin D3 agonist, p53 modulators, gene therapies, and mixtures of two or more thereof.

In yet another embodiment, the conventional cancer treatment agents that are suitable for use with the present invention include those agents specifically disclosed in Table 1.

Table 1 also provides known median dosages for selected chemotherapeutic agents. Specific dose levels for these agents will necessarily depend upon considerations such as those identified herein for the compounds of the present invention.

TABLE 1

| CHEMOTHERAPEUTIC AGENT | MEDIAN DOSAGE |
|---|---|
| Asparaginase | 10,000 units |
| Bleomycin Sulfate | 15 units |
| Carboplatin | 50-450 mg |
| Carmustine | 100 mg |
| Cisplatin | 10-50 mg |
| Cladribine | 10 mg |
| Cyclophosphamide (lyophilized) | 100 mg-2 gm |
| Cyclophosphamide (non-lyophilized) | 100 mg-2 gm |
| Cytarabine (lyophilized powder) | 100 mg-2 gm |
| Dacarbazine | 100 mg-200 mg |
| Dactinomycin | 0.5 mg |
| Daunorubicin | 20 mg |
| Diethylstilbestrol | 250 mg |
| Doxorubicin | 10-150 mg |
| Etidronate | 300 mg |
| Etoposide | 100 mg |
| Floxuridine | 500 mg |
| Fludarabine Phosphate | 50 mg |
| Fluorouracil | 500 mg-5 gm |
| Goserelin | 3.6 mg |
| Granisetron Hydrochloride | 1 mg |
| Idarubicin | 5-10 mg |
| Ifosfamide | 1-3 gm |
| Leucovorin Calcium | 50-350 mg |
| Leuprolide | 3.75-7.5 mg |
| Mechlorethamine | 10 mg |
| Medroxyprogesterone | 1 gm |
| Melphalan | 50 gm |
| Methotrexate | 20 mg-1 gm |
| Mitomycin | 5-40 mg |
| Mitoxantrone | 20-30 mg |
| Ondansetron Hydrochloride | 40 mg |
| Paclitaxel | 30 mg |
| Pamidronate Disodium | 30-*90 mg |
| Pegaspargase | 750 units |
| Plicamycin | 2,500 mcgm |
| Streptozocin | 1 gm |
| Thiotepa | 15 mg |
| Teniposide | 50 mg |
| Vinblastine | 10 mg |
| Vincristine | 1-5 mg |
| Aldesleukin | 22 million units |
| Epoetin Alfa | 2,000-10,000 units |
| Filgrastim | 300-480 mcgm |
| Immune Globulin | 500 mg-10 gm |
| Interferon Alpha-2a | 3-36 million units |
| Interferon Alpha-2b | 3-50 million units |
| Levamisole | 50 mg |
| Octreotide | 1,000-5,000 mcgm |
| Sargramostim | 250-500 mcgm |

The present invention can also be used in conjunction with immunotherapies. Not only may the methods and compositions herein disclosed be used with the increasing variety of immunological reagents now being tested or used to treat cancer, but it also may be used with those that come into practice in the future. The present invention thus may be used with immunotherapies based on polyclonal or monoclonal antibody-derived reagents, for instance. Such reagents are described in, for instance, Monoclonal Antibodies—Production, Engineering And Clinical Applications, Ritter et al., Eds., Cambridge University Press, Cambridge, UK (1995), which is incorporated by reference herein in its entirety. Radiolabelled monoclonal antibodies for cancer therapy, in particular, also are well known and are described in, for instance, Cancer Therapy With Radiolabelled Antibodies, D. M. Goldenberg, Ed., CRC Press, Boca Raton, Fla. (1995), which is incorporated by reference herein in its entirety.

Immunotherapy may also be achieved by combining antibodies with immunotoxins such as ricin. Immunotoxins may be bound to antibodies which, when combined, recognize the acute lymphoblastic leukemia and provide a more concentrated and targeted treatment of the acute lymphoblastic leukemia. This targeted therapy is also known as toxin therapy.

Complement proteins may also be used as an immunotherapy in conjunction with the present invention. The complement system consists of a series of proteins that work to “complement” the work of antibodies in destroying bacteria. Complement proteins circulate in the blood in an inactive form. The so-called “complement cascade” is set off when the first complement molecule encounters antibody bound to antigen in an antigen-antibody complex. Each of the complement proteins performs its specialized job in turn, acting on the molecule next in line. The end product is a cylinder that punctures the cell membrane and, by allowing fluids and molecules to flow in and out, dooms the target cell.

When combined with the antibodies that recognize the acute lymphoblastic leukemia, the complement proteins provide a more concentrated and targeted treatment of the acute lymphoblastic leukemia.

Cryotherapy recently has been applied to the treatment of some cancers. Methods and compositions of the present invention also can be used in conjunction with an effective therapy of this type.

According to another aspect of the invention, pharmaceutical compositions of matter useful for treating acute lymphoblastic leukemia are provided that contain, in addition to the aforementioned compounds, an additional therapeutic agent. Such agents may be chemotherapeutic agents, ablation or other therapeutic hormones, antineoplastic agents, monoclonal antibodies useful against cancers and angiogenesis inhibitors. A wide variety of other effective agents also may be used.

According to another aspect of the invention, the pharmaceutical composition of the present invention is useful for the therapy and prophylaxis of acute lymphoblastic leukemia. In prophylactic applications, compositions containing the present invention are administered to a patient susceptible to or otherwise at risk for acute lymphoblastic leukemia. Such an amount is defined to be a “prophylactically effective dose.” In this use, the precise amounts again depend on the patient's state of health and weight.

As used herein, the terms “therapeutic response time” mean the duration of time that a compound is present or detectable within a subject's body.

As used herein, the terms “treating” or “to treat,” mean to alleviate symptoms, eliminate the causation either on a temporary or permanent basis, or to prevent or slow the appearance of symptoms. The term “treatment” includes alleviation, elimination of causation of or prevention of a cardiovascular-related disorder associated with, but not limited to, any of the diseases or disorders described herein.

In another embodiment, the present invention is directed to the ALL-associated cell surface antigen recognized by the monoclonal antibodies described herein. More particularly, the present invention has identified and the target antigen of the γδ T cell receptor. This ALL-associated cell surface antigen has not been associated with cancer, tumor metastasis or ALL prior to its discovery by the present invention.

In accordance with the present invention, there is a specific population of γδ+ T cells that recognize a cell surface antigen restricted to ALL. Since Vδ1+ cells are activated against ALL in a non-MHC dependent manner, they recognize a specific ALL-associated surface antigen. Moreover, γδ+ T cells mediate non-MHC restricted cytolysis. See Fisch, P., et al J. Exp. Med. 171: 1567 (1990). They do not proliferate in the allogeneic one-way MLC, but do proliferate in response to primary ALL.

Survival of patients transplanted for ALL is greatly enhanced when increased numbers of Vδ1+ T cells are present during post-bone marrow transplant (BMT) recovery. Also, there is a specific cell-cell interaction between Vδ1+ cells and ALL. Therefore, a specific population of γδ+ T cells recognizes a cell surface antigen restricted to ALL.

Two widely known antigens that stimulate Vδ1+ cells are CD48 and the MICA/MICB antigens. See Steinle, A., et al., Proc. Nat. Acad. Sci. USA 95: 12510 (1998). CD48 is widely expressed on hematopoietic cells and has not been shown to be associated specifically with ALL stimulatory to γδ+ T cells. ALL cells that stimulate Vδ1+ cell proliferation have also not been found to express significant HSP-60 or HSP-70.

Interactions between B cells and γδ+ T cells have been reported. Vδ1 CTL expansion in HIV infection is accompanied by depletion of the Vδ2 subset and up-regulation of perforin-based cytolytic activity. Taken together with what is currently known about Vδ1 activation, the present invention has discovered that there is a ligand unique from the ones discussed above, which are responsible for ALL-induced Vδ1 expansion.

Identification of the ALL-associated cell surface antigen recognized by the γδ T cell receptor and described herein resulted from examining the differences between cells that activate γδ+ T cells and cells that do not. Thus, the present invention also encompasses additional screening methods for the isolation of other antigens, either entirely novel, or yet uncharacterized, as specific to ALL.

As described above, primary ALL stimulates expansion of γδ+ T cells in vitro as indeed do a number of cell lines derived from patients with pre-B ALL, with one notable exception. The JM1 cell line was derived from a patient with immunoblastic B cell lymphoma/leukemia and expresses B-ALL antigens CD19, CD10, and HLA-DR. Although phenotypically characteristic of B-ALL, the JM1 cell line is unable to stimulate any significant expansion of such cells under the same culture conditions, suggesting that they do not express the antigen or antigens recognized by γδ+ T cells. As such, they are ideally suited as a point of reference with which to compare differential gene expression patterns in populations of ALL cells that do stimulate a γδ+ T cell response. They also represent a vehicle within which to perform functional screening of additional candidate antigens in order to identify those that activate γδ+ T cells.

In another embodiment the present invention encompasses several methods and assays for the study of differential gene expression in order to identify, isolate and purify antigens responsible for γδ+ T cell responses. For example, the present invention provides methods and assays based on subtractive hybridization and proteomics. Other approaches include, but are not limited to, several molecular techniques that study differential gene expression such as array-based transcriptional profiling, differential display, and subtractive hybridization.

In yet another embodiment, the use of array-based transcriptional profiling allows the survey of the genes expressed by ALL that stimulate the proliferation of γδ+ T cells by comparison of the expression profiles of non-stimulatory JM1 cells. Arrays containing large numbers of cDNA molecules corresponding to individual expressed mRNA's within different types of cells allows for the differential comparison of the expressed genes within each.

The present invention also provides a differential display method for ALL-associated antigen screening. Differential display is a technique in which radiolabeled cDNA products of semi-random primed reverse transcriptions from two or more mRNA templates under comparison are resolved by high resolution gel electrophoresis and compared, Differentially expressed genes are indicated by the presence of a discretely sized cDNA product generated from one RNA template not represented among the products generated from the other RNA template(s). Differential display has emerged as a powerful tool with which to study differential gene expression, since it is a bidirectional technique capable of detecting both up and down-regulation of gene expression. Differential display also has the ability to identify entirely novel differentially expressed transcripts. Because of this latter feature, the present invention provides a differential display method as an ALL-associated antigen hunting technique.

Once differentially expressed genes have been detected, each differentially expressed band is then excised, sequenced, and as appropriate, subjected to full length cloning. Afterwards, the candidate genes be tested for their ability to confer a phenotype stimulatory to γδ+ T cells, upon non-stimulatory JM-1 cells.

In still another embodiment, a novel method for the isolation of genes differentially expressed by immunogenic ALL blast cells by subtractive hybridization is utilized as a screen for ALL-associated antigens. This method is then followed by simultaneous screening of all differentially expressed transcripts contained within a candidate pool of minimal complexity, using gene-modified JM1 cells challenged with γδ+ T cells to identify immunogenic clones.

Subtractive hybridization is based upon the hybridization of single-stranded cDNA, reverse transcribed from mRNA templates derived from a control population of cells (subtractor cells), with mRNA transcripts isolated from a second, and preferably closely-related, population of cells (target cells) thought to differentially express genes of interest. This is followed by the physical removal of hybrids that contain complementary mRNA and cDNA molecules, representing non-differentially expressed transcripts common to both target and subtractor populations, respectively. Having removed mRNA:cDNA hybrids, mRNA transcripts differentially expressed by target cells can then be used to generate probes with which to screen cDNA libraries, or reverse transcribed to allow construction libraries of differentially expressed genes. In common with differential display, subtractive hybridization can lead to the identification of novel genes, albeit in a unidirectional manner, but unlike differential display, this technique can also result in the direct isolation of full-length differentially expressed genes. This constitutes a major advantage over differential display within the context of functional screening.

In one embodiment, the detection of mRNA can be determined by a variety of methods well known to those of skill in the art, which can be carried out using well known and readily available starting materials, including those widely available from commercial suppliers.

A given mRNA may be determined in cells of sample tissue by in situ hybridization to a specific probe. Such probes may be cloned DNAs or fragments thereof, RNA, typically made by in vitro transcription, or oligonucleotide probes, usually made by solid phase synthesis. Methods for making and using probe suitable for specific hybridization in situ are ubiquitously known and used in the art.

By specific hybridization is meant that the probe forms a duplex with the given, target mRNA that is stable to the conditions of hybridization and subsequent incubations and that duplexes formed between the probe and other, non-target mRNAs are not stable and generally do not persist through subsequent incubations. Specific hybridization, thus means that the ratio of hybridization to target and non-target mRNAs provides an accurate determination of the target mRNA in cells in the sample.

In general, a sample is obtained by suitable surgical procedure and snap frozen, as by freezing in methybutane/dry ice. The samples can be embedded and sectioned much as described above for the determination of protein in samples. Sections can be thawed onto and affixed to glass slides previously cleaned with acid and ethanol and coated with poly-L-lysine. The tissue sections thereafter can be exposed to buffered formaldehyde, acetylated, treated with buffered glycine and then prehybridized in 50% formamide, 2×SSC (where 1×SSC is 0.15M NaCl, 0.015M sodium citrate, pH 7.0). After prehybridization the sections can be hybridized to the labeled probe in 50% formamide, 10% dextran sulfate, 2×SSC.

The exact conditions of the steps in the procedure, especially the prehybridization, hybridization and criterion steps will be adjusted with the T.sub.m (or the T.sub.d) of the probe and to provide the desired degree of specificity of hybridization; i.e., the desired stringency.

Theoretical approximations and empirical methods for determining proper conditions in this regard are well known and routinely practiced by those skilled in the pertinent arts. Approximation calculations and experimental techniques in this regard are described, for instance, in Sambrook, J. et al., Molecular Cloning: A Laboratory Manual, 2nd Ed., Cold Spring Harbor Laboratory Press, Cold Spring Harbor, N.Y. (1989).

Those of skill will appreciate, for instance, that the formamide in the foregoing solutions serves to provide equivalent hybridization conditions at lower temperature. For instance, hybridization in 50% formamide at about 50.degree. C. provides conditions similar to hybridization at 65.degree. C. without formamide. The lower temperature of hybridization can help preserve the sample sections during the hybridization procedure, aiding subsequent identification and examination of cells and mRNA content. Other agents that preserve features of the tissue sections that aid analysis likewise are preferred.

Dextran sulfate generally is used to accelerate the hybridization reaction and to drive it to completion in a shorter period of time, as is well known. Similar agents that increase the rate of hybridization, consistent with accurate determination of specific mRNA content, also are useful in the present invention.

Following hybridization, the probe-containing solution and unbound probe are removed. Typically, the sections are washed several times with prehybridization buffer, such as 50% formamide, 2×SSC, at or slightly above the hybridization temperature.

If an RNA probe is used for detection of the target mRNA, the sections then are treated with RNAseA, typically in the same solution, and then washed to remove RNAseA and byproducts with 50% formamide, 2×SSC under the same conditions as the previous washings. Finally, the sections typically are washed several additional times in 2×SSC at room temperature and then air-dried.

Radioactive probes generally are visualized by autoradiography. For this purpose slides can be dipped in a photographic emulsion, dried and allowed to expose the emulsion at 4.degree. C. for an appropriate period of time. Using a preferred emulsion, NTB-2 nuclear track emulsion, exposure times of 3 to 7 days are appropriate. The exposure time can be altered by a variety of factors including the use of more highly labelled probes.

The emulsions are developed at the end of the exposure period and then, typically, counterstained with hematoxylin and eosin. Subsequently, labelling of target mRNA in cells can be assessed by microscopy using brightfield and darkfield illumination.

A variety of controls may be usefully employed to improve accuracy in assays of this type. For instance, sections may be hybridized to an irrelevant probe and sections may be treated with RNAseA prior to hybridization, to assess spurious hybridization.

In one embodiment, techniques employed to assess restriction fragment length polymorphisms (“RFLP”) are applied to detect some mutations associated with aberrant splicing patterns. The assessment always can be made on the mRNA, but in some dysfunctions, it can be made on the genomic DNA as well. The mRNA or DNA can be amplified prior to RFLP analysis, as well, using PCR or other suitable technique.

In addition to RFLP techniques, SSCP can be used to detect aberrant splicing of messages. For this purpose, a target mRNA is amplified by reverse transcriptase-mediated PCR. The double-stranded amplified DNA is denatured and run on gels in which mobility is quite sensitive to small changes in secondary structure.

Yet another technique that can be employed to determine aberrant splicing, among other things, is ligase mediated PCR. This technique also is well known to those of skill in the art, and techniques suitable to the analysis and determination of mRNAs and genomic aberrations that have been described in the literature readily can be applied to the determination of aberrant mRNAs in cancer cells.

In another embodiment, the present invention provides a novel composition comprising a monoclonal antibody that is specific for at least one epitope of the ALL-associated cancer antigen. The antibody can be prepared by hybridoma fusion techniques or by techniques that utilize EBV-immortalization technologies.

In another embodiment, the present invention encompasses a pharmaceutical composition comprising an isolated antibody, which specifically binds to an epitope of an ALL-associated cell surface antigen, and a pharmaceutically acceptable carrier.

In still another embodiment, the present invention encompasses a pharmaceutical composition comprising an isolated antibody which specifically binds to an epitope of an ALL-associated cell surface antigen, and a pharmaceutically acceptable carrier, wherein said antigen is capable of being recognized by a γδ+ T cell receptor having a partial polynucleotide sequence comprising SEQ ID NO: 1.

In still another embodiment, the present invention encompasses a pharmaceutical composition comprising an isolated antibody which specifically binds to an epitope of an ALL-associated cell surface antigen, and a pharmaceutically acceptable carrier, wherein said antibody is a monoclonal antibody.

In still another embodiment, the present invention encompasses a pharmaceutical composition comprising an isolated antibody which specifically binds to an epitope of an ALL-associated cell surface antigen, and a pharmaceutically acceptable carrier, wherein said antibody is a polyclonal antibody.

In still another embodiment, the present invention encompasses a pharmaceutical composition comprising an isolated antibody which specifically binds to an epitope of an ALL-associated cell surface antigen, and a pharmaceutically acceptable carrier, wherein said antibody specifically binds to the same ALL-associated cell surface antigen as the monoclonal antibody produced by the hybridoma cell line having ATCC Accession No. #.

The term “antibody” as used herein, unless indicated otherwise, is used broadly to refer to both antibody molecules and a variety of antibody derived molecules. Such antibody derived molecules comprise at least one variable region (either a heavy chain of light chain variable region) and include molecules such as Fab fragments, Fab′ fragments, F(ab′).sub.2 fragments, Fd fragments, Fab′ fragments, Fd fragments, Fabc fragments, Sc antibodies (single chain antibodies), diabodies, individual antibody light chains, individual antibody heavy chains, chimeric fusions between antibody chains and other molecules, and the like.

Polyclonal antibodies are also encompassed by the present invention and are prepared according to standard protocols in the art by injecting the antigen of interest into various animals, such as, for example, goats and rabbits, and then subsequently purifying the antibodies from the animal's serum. The procedure for making antibodies is well known in the art and can be found in, e.g., Harlow, E. and Lane, D., Antibodies: A Laboratory Manual, Cold Spring Harbor Press (1988).

The hybridoma fusion techniques of the present invention are provided by, for example, Kohler and Milstein. See, Kohler and Milstein, Nature, 256: 495-97 (1975); Brown et al., J. Immunol., 127 (2): 539-46 (1981); Brown et al., J. Biol. Chem., 255: 4980-83 (1980); Yeh et al., Proc. Nat'l. Acad. Sci. (USA), 76 (6):2927-31 (1976); and Yeh et al., Int. J. Cancer, 29: 269-75 (1982).

These techniques involve the injection of an immunogen (e.g., purified antigen or cells or cellular extracts carrying the antigen) into an animal (e.g., a mouse) so as to elicit a desired immune response (i.e., production of antibodies) in that animal. For example, γδ+ T cells, whole or in part, or the ALL-associated cell surface antigen, whole or in part, are used as the immunogen. The immunogen preparation is injected, for example, into a mouse, and after a sufficient time the mouse is sacrificed and somatic antibody-producing lymphocytes are obtained.

Antibody-producing cells are derived from the lymph nodes, spleens and peripheral blood of primed animals. Spleen cells are preferred. Mouse lymphocytes give a higher percentage of stable fusions with the mouse myelomas described below. The use of rat, rabbit and frog somatic cells is also encompassed by the present invention. The spleen cell chromosomes encoding desired immunoglobulins are immortalized by fusing the spleen cells with myeloma cells, generally in the presence of a fusing agent such as polyethylene glycol (PEG). Any of a number of myeloma cell lines is used as a fusion partner according to standard techniques; for example, the P3-NS1/1-Ag4-1, P3-x63-Ag8.653 or Sp2/O-Ag14 myeloma lines. These myeloma lines are available from the American Type Culture Collection (ATCC), Rockville, Md.

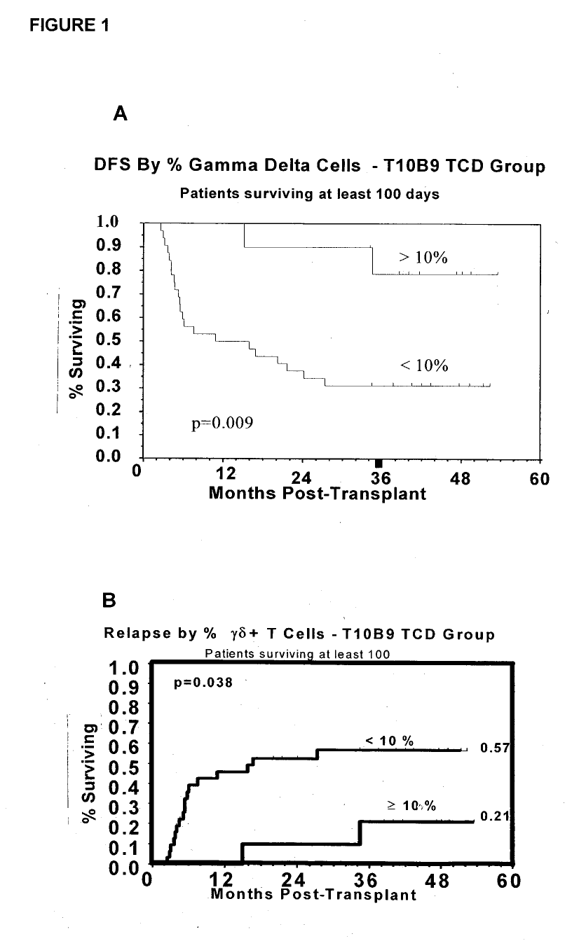

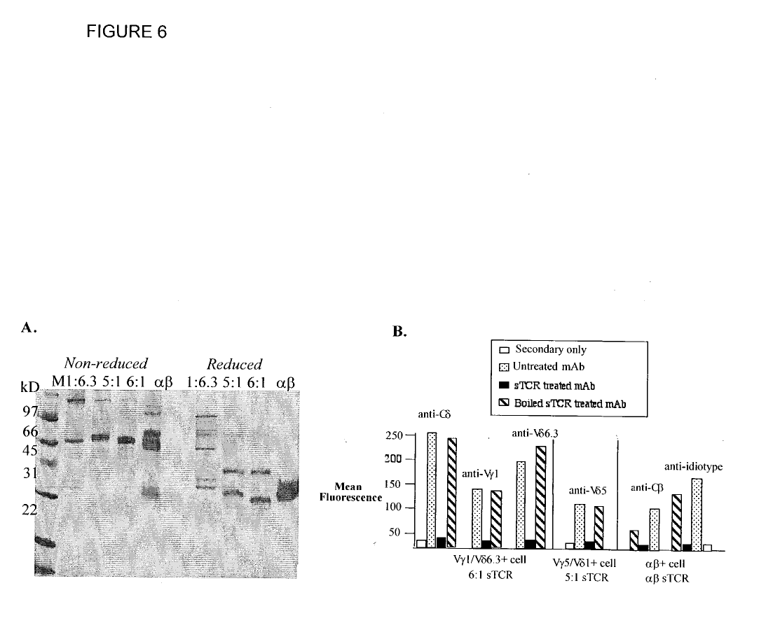

The resulting cells, which include the desired hybridomas, are then grown in a selective medium, such as HAT medium, in which unfused parental myeloma or lymphocyte cells eventually die. Only the hybridoma cells survive and are grown under limiting dilution conditions to obtain isolated clones. The supernatants of the hybridomas are screened for the presence of antibody of the desired specificity, e.g., by immunoassay techniques using the antigen that has been used for immunization. Positive clones are then subcloned under limiting dilution conditions, and the monoclonal antibody produced is isolated. Various conventional methods exist for isolation and purification of the monoclonal antibodies so as to free them from other proteins and other contaminants. Commonly used methods for purifying monoclonal antibodies include ammonium sulfate precipitation, ion exchange chromatography, and affinity chromatography. See, e.g., Monoclonal Hybridoma Antibodies: Techniques and Applications, Hurell (ed.), Zola et al., 51-52, CRC Press (1982). Hybridomas produced according to these methods are propagated in vitro or in vivo (in ascites fluid) using techniques known in the art. See, generally, Fink et al., supra, at page 123, FIGS. 6-1.

Generally, the individual cell line is propagated in vitro, for example in laboratory culture vessels, and the culture medium containing high concentrations of a single specific monoclonal antibody is harvested by decantation, filtration or centrifugation. Alternatively, the yield of monoclonal antibody is enhanced by injecting a sample of the hybridoma into a histocompatible animal of the type used to provide the somatic and myeloma cells for the original fusion. Tumors secreting the specific monoclonal antibody produced by the fused cell hybrid develop in the injected animal. The body fluids of the animal, such as ascites fluid or serum, provide monoclonal antibodies in high concentrations. When human hybridomas or EBV-hybridomas are used, it is necessary to avoid rejection of the xenograft injected into animals such as mice. Immunodeficient or nude mice are used or the hybridoma is passaged first into irradiated nude mice as a solid subcutaneous tumor, cultured in vitro and then injected intraperitoneally into pristane primed, irradiated nude mice which develop ascites tumors secreting large amounts of specific human monoclonal antibodies.

For certain therapeutic applications chimeric (mouse-human) or human monoclonal antibodies are preferable to murine antibodies because patients treated with mouse antibodies generate human antimouse antibodies. See Shawler et al., J. Immunol., 135: 1530-35 (1985). Chimeric mouse-human monoclonal antibodies reactive with the ALL-associated antigen can be produced, for example, by techniques recently developed for the production of chimeric antibodies. See Oi et al., Biotechnologies, 4 (3): 214-221 (1986) and Liu et al., Proc. Nat'l. Acad. Sci. USA, 84: 3439-43 (1987). Chimeric antibodies and methods for their production are known in the art. See, e.g., Cabilly et al., European Patent Application 125023 (published Nov. 14, 1984); Taniguchi et al., European patent Application 171496 (published Feb. 19, 1985); Morrison et al., European Patent Application 173494 (published Mar. 5, 1986); Neuberger et al., PCT Application WO 86/01533, (published Mar. 13, 1986); Kudo et al., European Patent Application 184187 (published Jun. 11, 1986); Robinson et al., International Patent Publication #PCT/US86/02269 (published May 7, 1987); Liu et al., Proc. Natl. Acad. Sci. USA 84: 3439-3443 (1987); Sun et al., Proc. Natl. Acad. Sci. USA 84: 214-218 (1987); and Better et al., Science 240: 1041-1043 (1988).

Generally, DNA segments encoding the H and L chain antigen-binding regions of the murine mAb can be cloned from the mAb-producing hybridoma cells, which can then be joined to DNA segments encoding C.sub.H and C.sub.L regions of a human immunoglobulin, respectively, to produce murine-human chimeric immunoglobulin-encoding genes.

Humanized antibodies can be made using a second approach, i.e., to construct a reshaped human antibody, which has been described in, e.g, See Maeda et al., Hum. Antibod. Hybridomas 2: 124-134 (1991) and Padlan, Mol. Immunol. 28: 489-498 (1991). As used herein, the term “humanized” antibody refers to a molecule that has its CDRs (complementarily determining regions) derived from a non-human species immunoglobulin and the remainder of the antibody molecule derived mainly from a human immunoglobulin.

The tumor antigen and the antigenic cancer epitopes thereof may be purified and isolated from natural sources such as from primary clinical isolates, cell lines and the like. The cancer peptides and their antigenic epitopes may also be obtained by chemical synthesis or by recombinant DNA techniques known in the arts. Techniques for chemical synthesis are described in Steward et al. (1969); Bodansky et al. (1976); Meienhofer (1983); and Schroder et al. (1965).

Accordingly, genes coding for the constant regions of the murine ALL-associated antigen antibody molecule are substituted with human genes coding for the constant regions of an antibody with appropriate biological activity. Novel antibodies of mouse or human origin, can also be made to the antigen having the appropriate biological functions. For example, human monoclonal antibodies are made by using the antigen of the invention, to sensitize human lymphocytes to the antigen in vitro followed by EBV-transformation or hybridization of the antigen-sensitized lymphocytes with mouse or human lymphocytes as previously described. See Borrebaeck et al. Proc. Nat'l. Acad. Sci. USA, 85: 3995-99 (1988).

According to one embodiment, the antibody of this invention, designated “ALL-associated cell surface antigen”, was produced via hybridoma techniques using an antigen from the surface of ALL blasts that specifically binds to the γδ T cell receptor, which has the partial polynucleotide sequence according to SEQ ID NO.1. The hybridoma cell line, producing the ALL-associated cell surface antigen-specific antibody, has been deposited with the American Type Culture collection (ATCC), 12301 Parklawn Drive, Rockville, Md. 20852, on ##, and has there been identified as follows:

#: Accession No.: HB ####.

The present invention also provides several immunotherapy methods for treating a neoplasia disorder that can be divided into active or passive categories. Active immunotherapy involves the direct immunization of cancer patients with cancer antigens in an attempt to boost immune responses against the tumor. Passive immunotherapy refers to the administration of immune reagents, such as immune cells or antibodies with antitumor reactivity with the goal of directly mediating antitumor responses.

Therefore, in another embodiment, the antibody composition of the present invention is also useful as a vaccine to prevent or treat cancer. Likewise, another aspect of the invention is a vaccine useful in inducing tumor-specific cell-mediated immunity against cancer.

The antigen composition may further comprise at least one co-immunostimulatory molecule. Co-immunostimulatory molecules to be used in conjunction with the tumor antigen of the present invention for stimulating antigen specific T cell responses include, but are not limited to, one or more major histocompatibility complex (MHC) molecules, such as class I and class II molecules, preferably a class I molecule. DNA sequences of MHC co-immunostimulatory molecules are available from DNA sequence repositories, such as GenBank and the like. The composition may further comprise other stimulator molecules including B7.1, B7.2, ICAM-1, ICAM-2, LFA-1, LFA-3, CD72 and the like, and cytokines which include but are not limited to IL-1 through IL-15, TNF.alpha., IFN.γ., RANTES, G-CSF, M-CSF, IFN.alpha., CTAP III, ENA-78, GRO, I-309, PF-4, IP-10, LD-78, MGSA, MIP-1.alpha., MIP-1.beta., or combination thereof, and the like for immunopotentiation.

The vaccine can comprise the entire isolated and purified ALL-associated cell surface antigen, a subpart of the antigen, the antigen on a cell surface of an antigen presenting cell such as B cell or macrophage or dendritic cell, in the membrane of a liposome, or expressed on the surface of a transduced or transfected cell, or an epitope specific to the antigen.

In another embodiment, the ALL-associated cell surface antigen, cancer peptides or variants thereof may be in the form of a derivative in which other constituents are attached thereto such as radiolabels, biotin, fluorescein. A targeting agent may also be attached to the tumor antigen or cancer peptides that allow for specific targeting to a specific organ, tumor or cell types. Such targeting agents may be hormones, cytokines, cellular receptors and the like.

The ALL-associated cell surface antigen of the present invention is also useful in a diagnostic assay for detecting cancer or precancer, including such cancers as ALL, in a mammal.

Thus, the present invention also encompasses a novel method for diagnosing a neoplasia disorder in a mammal, wherein said neoplasia disorder produces a ALL-associated cell surface antigen comprising:

a) providing a sample of biological material from said mammal;

b) contacting said biological material with antibodies specific for the antigen;

c) detecting the presence or absence of an immunological reaction product between said antibodies and said ALL-associated cell surface antigen, the presence of an immunological reaction product being indicative of said neoplasia disorder in said mammal.

For purposes of diagnosing whether a particular subject has a cancer or a precancer, a sample is taken from the subject, e.g., a biopsy specimen taken from tissue suspected of having a metastatic tumor. Generally, the sample is treated before an assay is performed. Assays, which are suitable for purposes of the present invention, include ELISA, RIA, EIA, Western Blot analysis, immunohistological staining, and the like. Furthermore, depending upon the assay used, the antigens or the antibodies of the present invention can be labeled by an enzyme, a fluorophore or a radioisotope. See, e.g., Current Protocols in Immunology, Coligan et al., John Wiley & Sons Inc., New York, N.Y. (1994) and Frye et al., Oncogen 4: 1153-1157 (1987).

As is known in the art, the treatment of the sample may vary depending on the assay that is used to detect an antigen. For example, cells of tissue biopsy can be lysed and the cell lysates are used in e.g., Western Blot analysis. For assays such as the Whole Cell ELISA assay, cells can be washed with, e.g., PBS, and then fixed with 0.25% glutaraldehyde in PBS before the assay.

In other embodiments, a cancer can be diagnosed by detecting the expression of the ALL-associated cell surface antigen through the use of a nucleic acid probe, e.g., probes that hybridize to the mRNA of the antigen. For example, a nucleic acid probe can be made from any region of the ALL-associated cell surface antigen's RNA or DNA. A probe for detecting the mRNA of the target antigen of the γδ T cell receptor can be made based on the polynucleotide sequence of such antigen. In order to produce a detectable signal, the probe is generally at least about 14 nucleotides, and labeled with either an enzyme (such as horse radish peroxidase, alkaline phosphatase, glucose oxidase and beta.-galactosidase), a fluorophore or a radioisotope.

Detection of the expression of an antigen with a nucleic acid probe can be achieved by a variety of hybridization procedures, which are well known in the art. For example, mRNA can be extracted from tissue specimen and analyzed in, e.g., Northern Blot analysis. Alternatively, in situ hybridization procedure can be employed, in which lysis of cells and isolation of RNA is not necessary. See, e.g., Current Protocols in Molecular Cloning, Ausubel et al., John Wiley & Sons, New York.

A purified antibody specifically reactive with an immunoreactive epitope specific to the antigen is also provided. The term “reactive” means capable of binding or otherwise associating nonrandomly with an antigen. “Specific” immunoreactivity as used herein denotes an antigen or epitope (amino acid, protein, peptide or fragment) that does not cross react substantially with an antibody that is immunoreactive with other antigens.

The present invention further provides a kit for detecting the antigen. Particularly, the kit can detect the presence of an antigen specifically reactive with the antibody or an immunoreactive fragment thereof. The kit can include an antibody bound to a substrate, a secondary antibody reactive with the antigen and a reagent for detecting a reaction of the secondary antibody with the antigen. Such a kit can be an ELISA kit and can comprise the substrate, primary and secondary antibodies when appropriate, and any other necessary reagents such as detectable moieties, enzyme substrates and color reagents as described above. The diagnostic kit can, alternatively, be an immunoblot kit generally comprising the components and reagents described herein.

The diagnostic kit of the present invention can alternatively be constructed to detect nucleotide sequences specific for the antigen comprising the standard kit components such as the substrate and reagents for the detection of nucleic acids. Because neoplastic cells and antigen-stimulated cells can be diagnosed by detecting nucleic acids specific for the antigen in tissue and body fluids such as urine, saliva and serum, it will be apparent to one of skill in the art that a kit can be constructed that utilizes the nucleic acid detection methods, such as specific nucleic acid probes, primers or restriction fragment length polymorphs in analyses. It is contemplated that the diagnostic kits will further comprise a positive and negative control test.

The particular reagents and other components included in the diagnostic kits of the present invention can be selected from those available in the art in accord with the specific diagnostic method practiced in the kit. Such kits can be used to detect the antigen in tissue and fluid samples from a subject.

An isolated immunogenically specific epitope or fragment of the antigen is also provided. A specific immunogenic epitope of the antigen can be isolated from the whole antigen by chemical or mechanical disruption of the molecule. The purified fragments thus obtained can be tested to determine their immunogenicity and specificity by the methods taught herein. Immunoreactive epitopes of the antigen can also be synthesized directly. An immunoreactive fragment is defined as an amino acid sequence of at least about 5 consecutive amino acids derived from the antigen amino acid sequence.

By the identification of the antigen, the invention also encompasses the nucleotide sequence encoding the antigen. The polynucleotide sequence encoding for the antigen can be determined by standard procedures. For example, the amino terminal sequence of the antigen can be determined and a corresponding nucleotide sequence can be deduced. This nucleotide sequence can then be used to make a probe to hybridize sequences from a gene library.

In yet other embodiments, the ALL-associated cell surface antigen or antibody composition may be prepared in the form of a kit, alone or in combination with other reagents.

In still other embodiments, the present invention provides a kit for diagnosing or monitoring a cancer disorder which said cancer disorder produces a ALL-associated cell surface antigen that is specifically bound by a γδ T cell receptor comprising:

a) providing a sample of biological material from said mammal wherein the biological material is plasma, serum, cytosol fluid, ascites or tissue;

b) contacting said biological material with antibodies specific for the antigen;

c) detecting the presence or absence of an immunological reaction product between said antibodies and said ALL-associated cell surface antigen, the presence of an immunological reaction product being indicative of or an early indication of said cancer disorder.

Thus, a diagnostic kit can also be used to perform the above described method.

In the present invention, a composition comprising an antibody that is specific for the ALL-associated cell surface antigen described herein is administered to a subject in need of such treatment according to standard routes of drug delivery that are well known to one of ordinary skill in the art.

The antibody can be supplied as a pure compound, or in the form of a pharmaceutically active salt. The antibody can also be supplied in the form of a prodrug, an isomer, a racemic mixture, or in any other chemical form or combination that, under physiological conditions, is still capable of binding to the ALL-associated cell surface antigen described herein.

Any non-toxic, inert and effective carrier may be used to formulate compositions of the present invention. Well known carriers used to formulate other therapeutic compounds for administration to humans particularly will be useful in the compositions of the present invention. Pharmaceutically acceptable carriers, excipients and diluents in this regard are well known to those of skill, such as those described in the Merck Index, 11th Ed., Budavari et al., Eds., Merck & Co., Inc., Rahway, N.J. (1989). Examples of such useful pharmaceutically acceptable excipients, carriers and diluents include distilled water, physiological saline, Ringer's solution, dextrose solution, Hank's solution and DMSO, which are among those preferred for use in the present invention.

Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

Suitable pharmaceutically-acceptable base addition salts of compounds of the present invention include metallic ion salts and organic ion salts. More preferred metallic ion salts include, but are not limited to appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

Antibodies that are useful in the present invention can be of any purity or grade, as long as the preparation is of a quality suitable for pharmaceutical use. The antibodies can be provided in pure form, or it can be accompanied with impurities or commonly associated compounds that do not affect its physiological activity or safety.

The antibodies can be provided in a pharmaceutically acceptable carrier or excipient to form a pharmaceutical composition. Pharmaceutically acceptable carriers and excipients include, but are not limited to, physiological saline, Ringer's solution, phosphate solution or buffer, buffered saline and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

Therapeutic treatment with the novel composition of the invention can utilize any type of administration including topical, other non-invasive and invasive means.

Administration by non-invasive means may be by oral, intranasal or transdermal routes, among others. Administration by invasive techniques may be intravenous, intraperitoneal, or intramuscular, among others.

Administration may be by a single dose, it may be repeated at intervals or it may be continuous. Where continuous administration is applied, infusion is preferred. In this situation, pump means often will be particularly preferred for administration. Especially, subcutaneous pump means often will be preferred in this regard.

The compositions and methods of the invention also may utilize controlled release technology. Thus, for example, the novel composition of the invention may be incorporated into a hydrophobic polymer matrix for controlled release over a period of days. Such controlled release films are well known to the art. Examples of polymers commonly employed for this purpose that may be used in the present invention include nondegradable ethylene-vinyl acetate copolymer and degradable lactic acid-glycolic acid copolymers. Certain hydrogels such as poly(hydroxyethylmethacrylate) or poly(vinylalcohol) also may be useful, but for shorter release cycles then the other polymer releases systems, such as those mentioned above.

The pharmaceutical compositions may be administered enterally and parenterally. Oral (intra-gastric) is a preferred route of administration. Pharmaceutically acceptable carriers can be in solid dosage forms for the methods of the present invention, which include tablets, capsules, pills, and granules, which can be prepared with coatings and shells, such as enteric coatings and others well known in the art. Liquid dosage forms for oral administration include pharmaceutically acceptable emulsions, solutions, suspensions, syrups, and elixirs.

Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in a mixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the antibodies described herein may be formulated with sweetening agents, for example glycerol, sorbitol, or sucrose. Such formulations may also contain a demulcent, a preservative and flavoring and coloring agents.

The subject method of prescribing an antibody that is specific for the ALL-associated cell surface antigen and compositions comprising the same can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

Administration can also be by inhalation, in the form of aerosols or solutions for nebulizers, or rectally, in the form of suppositories prepared by mixing the drug with a suitable non-irritating excipient which is solid at ordinary temperature, but liquid at the rectal temperature and will therefore, melt in the rectum to release the drug. Such materials are cocoa butter and polyethylene glycols.

Also encompassed by the present invention is buccal or “sub-lingual” administration, which includes lozenges or a chewable gum comprising the compounds, set forth herein. The compounds can be deposited in a flavored base, usually sucrose, and acacia or tragacanth, and pastilles comprising the compounds in an inert base such as gelatin and glycerin or sucrose and acacia.

Other methods for administration of the compositions of the present invention include dermal patches that release the medicaments directly into a subject's skin.

Topical delivery systems are also encompassed by the present invention and include ointments, powders, sprays, creams, jellies, collyriums, solutions or suspensions.

Preservatives are optionally employed to prevent microbial contamination during use. Suitable preservatives include: polyquaternium-1, benzalkonium chloride, thimerosal, chlorobutanol, methyl paraben, propyl paraben, phenylethyl alcohol, edetate disodium, sorbic acid, or other agents known to those skilled in the art. The use of polyquaternium-1 as the antimicrobial preservative is preferred. Typically, such preservatives are employed at a level of from 0.001% to 1.0% by weight.

Pharmaceutically acceptable excipients and carriers encompass all the foregoing and the like. The above considerations concerning effective formulations and administration procedures are well known in the art and are described in standard textbooks. See e.g. Remington: The Science and Practice of Pharmacy, 20^{th} Edition, Gennaro, A. R., Lippincott, Williams and Wilkins (2000); Remington's Pharmaceutical Sciences, Hoover, John E., Mack Publishing Co., Easton Pa., (1975); Pharmaceutical Dosage Forms, Liberman, et al., Eds., Marcel Decker, New York, N.Y. (1980); and Kibbe, et al., Eds., Handbook of Pharmaceutical Excipients (3^{rd} Ed.), American Pharmaceutical Association, Washington (1999).

The quantity of the active agent for effective therapy will depend upon a variety of factors, including the stage of acute lymphoblastic leukemia (ALL), means of administration, physiological state of the patient, other medicaments administered, and other factors.

Treatment dosages generally may be titrated to optimize safety and efficacy. Typically, dosage-effect relationships from in vitro studies initially will provide useful guidance on the proper doses for patient administration. Studies in animal models also generally may be used for guidance regarding effective dosages for treatment of cancer in accordance with the present invention.

These considerations, as well as effective formulations and administration procedures are well known in the art and are described in standard textbooks, such as Goodman And Gilman's: The Pharmacological Bases Of Therapeutics, 8th Ed., Gilman et al. Eds. Pergamon Press (1990) and Remington's Pharmaceutical Sciences, 17th Ed., Mack Publishing Co., Easton, Pa. (1990), both of which are incorporated by reference herein in their entirety.

In determining the effective amount or dose of the compositions of the present invention, a number of factors are considered by the attending diagnostician, including, but not limited to, the potency and duration of action of the compounds used, the nature and severity of the illness to be treated, as well as the sex, age, weight, general health and individual responsiveness of the patient to be treated, and other relevant circumstances.

As used herein, an “effective amount” means the dose or amount to be administered to a subject and the frequency of administration to the subject, which is readily determined by one having ordinary skill in the art, by the use of known techniques and by observing results obtained under analogous circumstances.

As used herein, the terms “prophylactically effective” refer to an amount of either an ALL-associated antigen-specific antibody or activated γδ+ T cells alone and in combination with conventional cancer treatment agents that causes a decrease in the frequency of incidence of neoplasia or an neoplasia-related disorder. The term “prophylactic” refers to the prevention of neoplasia or a neoplasia-related disorder, whereas the term “therapeutic” refers to the effective treatment of an existing neoplasia disorder.

It will be appreciated that the amount of the ALL-associated antigen-specific antibody or activated γδ+ T cells alone and in combination with conventional cancer treatment agents required for use in the treatment or prevention of neoplasia and neoplasia-related disorders will vary within wide limits and will be adjusted to the individual requirements in each particular case. In general, for administration to adults, an appropriate daily dosage is described herein, although the limits that are identified as being preferred may be exceeded if expedient. The daily dosage can be administered as a single dosage or in divided dosages.

Typical therapeutic doses of the pharmaceutical composition comprising the ALL-associated antigen-specific antibody are given at a dose between 1.0 μg/kg and 10 mg/kg, more preferably between 10 μg/kg and 5 mg/kg, most preferably between 0.1 and 2 mg/kg, based on the body weight of the subject.

Those skilled in the art will appreciate that dosages may also be determined with guidance from Goodman & Goldman's The Pharmacological Basis of Therapeutics, Ninth Edition, Appendix II, 1707-1711 (1996).

Any effective treatment regimen can be utilized and repeated as necessary to affect treatment. In clinical practice, the compositions containing the novel composition of the invention, alone or in combination with other therapeutic agents are administered in specific cycles until a response is obtained.

For patients who initially present without refractory acute lymphoblastic leukemia, drugs based on the novel composition of the invention can be used as an immediate initial therapy prior to surgery and radiation therapy, and as a continuous post-treatment therapy in patients at risk for recurrence of acute lymphoblastic leukemia.

For patients who initially present with refractory acute lymphoblastic leukemia, drugs based on the novel composition of the invention can be used as a continuous supplement to, or possible as a replacement for hormonal ablation.

In other embodiments, the present invention encompasses the use of gene therapy to treat a neoplasia-related disease. Currently, gene therapy protocols relate to therapy of certain carefully chosen disorders, including certain inherited disorders, a number of aggressively fatal cancers and AIDS. The restricted application of gene therapy to a few disorders reflects concerns about the efficacy, safety and ethical implications of the approach in general, and current techniques in particular. Despite the cautious approach mandated by these concerns, and despite the fact that techniques for carrying out gene therapy are still in an early stage of development, results from the first few trials have been very encouraging, some spectacularly so. It seems certain that gene therapy techniques will improve rapidly and that gene therapies soon will develop into an increasingly important and ubiquitous modality for treating disease. (Reviewed, for instance, in Tolstoshev, Ann. Rev. Pharm. Toxicol. 32: 573-596 (1993) and Morgan et al., Ann. Rev. Biochem. 62: 191-217 (1993), which are incorporated by reference herein in their entirety).

The delivery of a variety of therapeutic agents clearly will be accomplished by gene therapy techniques. Many of the procedures now in use or under current development for gene therapy may be used in accordance with the present invention to treat refractory acute lymphoblastic leukemia. Additional techniques that will be developed in the future similarly will be found useful in the present invention.

By gene therapy, in the following discussion, generally is meant the use of a polynucleotide, in a cell, to achieve the production of an agent and the delivery of the agent to engender a therapeutic effect in the patient. The agent may itself be a therapeutic agent or it may engender the production of a therapeutic agent upon introduction into the patient. Approaches to genetic therapy currently being developed, which can be used in accordance with this aspect of the invention disclosed herein, often are grouped into two major categories: ex vivo and in vivo techniques.

Ex vivo techniques generally proceed by removing cells from a patient or from a donor, introducing a polynucleotide into the cells, usually selecting and growing out, to the extent possible, cells that have incorporated, and, most often, can express the polynucleotide, and then introducing the selected cells into the patient.

In addition, the polynucleotide may be introduced directly into the patient. The polynucleotide in this case may be introduced systemically or by injection into the patient. The polynucleotide may be in the form of DNA or RNA, alone or in a complex, or in a vector. The polynucleotide may be in any of a variety of well-known forms, for instance, a DNA, a DNA fragment cloned in a DNA vector, a DNA fragment cloned in DNA vector and encapsidated in a viral capsid.

The polynucleotide may be an RNA or a DNA. More typically it is a DNA. It may include a promoter, enhancer and other cis-acting control regions that provide a desired level and specificity of expression in the cells of a region operably linked thereto that encodes an RNA, such as an anti-sense RNA, or a protein. The polynucleotides may contain several such operably linked control and encoding regions for expression of one or more mRNAs or proteins, or a mixture of the two.

The polynucleotide may be introduced into cells either ex vivo or in vivo. A variety of techniques have been designed to deliver polynucleotides into cells for constitutive or inducible expression, and these routine techniques can be used in gene therapy of the present invention as well. Polynucleotides will be delivered into cells ex vivo using cationic lipids, liposomes or viral vectors. Polynucleotides will be introduced into cells in vivo using direct or systemic injection. Methods for introducing polynucleotides in this manner can involve direct injection of a polynucleotide, which then generally will be in a composition with a cationic lipid or other compound or compounds with a cationic lipid or other compound or compounds that facilitate direct uptake of DNA by cells in vivo. Such compositions may also comprise ingredients that modulate physiological persistence. In addition, polynucleotides can be introduced into cells in vivo in viral vectors.

Genetic therapies in accordance with the present invention may involve a transient (temporary) presence of the gene therapy polynucleotide in the patient or the permanent introduction of a polynucleotide into the patient. In the latter regard, gene therapy may be used to repair a dysfunctional gene to prevent or inhibit metastasis. Genetic therapies may be used alone or in conjunction with other therapeutic modalities.

### EXAMPLES

The following examples are illustrative of the present invention and are not intended to be limitations thereon. Unless otherwise specified, all percentages are based on 100% by volume of the sample.

### Example 1

An increase in γδ+ T cells after BMT is associated with improved relapse-free survival. The presence of increased γδ+ T cells was first observed during a comprehensive study of immunophenotypic and functional recovery of peripheral blood lymphocytes following T cell depleted (TCD) allogeneic BMT. See Lamb, L. S., et al. Bone Marrow Transplantation 21: 471 (1998).

The recovery of γδ+ T cells was examined to explore the consequences of using a TCD monoclonal antibody specific to the αβ T cell receptor (T10B9.1A) that spares γδ+ T cells in the allogeneic graft. In most subjects, the percentage of peripheral blood γδ+ T cells was below 10 and the absolute count below 1.75×10⁵/ml throughout the post-BMT recovery period, consistent with normal proportions of αβ+ and γδ+ T cells.

It was noted that 10 leukemia patients who survived for at least 100 days following transplantation with αβ+ depleted haplodispirate grafts were found to have an increased (>10%) proportion of γδ+ T cells that first appeared between post-BMT day 60 and 270. Further analysis revealed no statistically significant differences with respect to patient age, donor age, race, sex mismatch, risk category, CMV status, T cell dose, HLA mismatch, infection, TBI dose, or disease type.

There was no significant difference in the incidence of acute or chronic GvHD between patients with γδ+ T cells ≦10% and those with >10% γδ T cells during post-BMT recovery. However, eight of these patients are surviving and are free of disease (FIG. 1A), as compared to a disease-free survival probability of 31% at 2.5 years among 100-day survivors with a normal proportion and concentration of γδ T cells (p=0.009). Eight of ten (80%) patients who developed a spontaneous increase in γδ+ T cells during the first year following BMT remain alive and free of disease for up to seven years (FIG. 1). Probability of relapse (FIG. 1B) in the high γδ+ T cell group was 21% vs 57% in the low/normal γδ+ T cell group (p=0.038).

FIG. 1 is a comparison of (A) disease free survival and (B) incidence of relapse from patients with increased γδ+ T cells post-BMT and patients with normal recovery of γδ+ T cells. FIG. 1 shows the significantly improved disease free survival and lower incidence of relapse in the group with increased γδ+ T cells.

No other factor was found to be associated with improved survival in patients with increased numbers of γδ+ T cells. See Lamb L. S., et al. J. Hematotherapy 5: 503 (1996).

Further studies have shown that enrichment of the marrow graft with γδ+ T cells contributed to the later development of increased γδ+ T cells, but the γδ+ T cells were protective regardless of the graft preparation method. See Lamb, L. S., et al. Cytotherapy 1: 7-19 (1999).

### Example 2

Patients with increased γδ+ T cells preferentially express the Vδ1 receptor while healthy donors and “normally” recovering patients show expression of multiple receptor subtypes. Healthy donors show predominant expression of Vδ2. These data suggest that the cells obtained form these patients have undergone an activation process that has resulted in Vδ1 expansion and the acquisition of cytolytic activity.

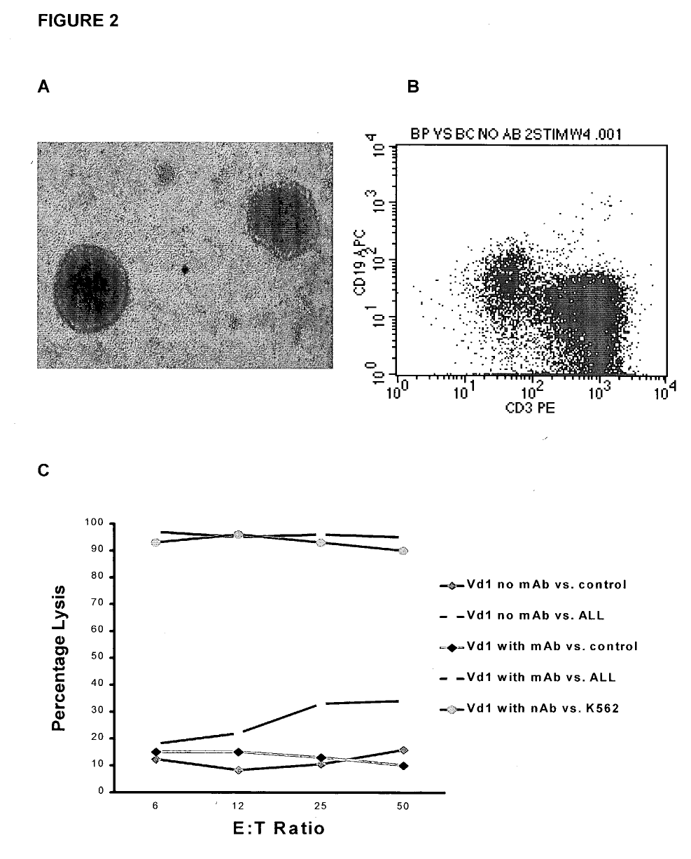

γδ+ T cells have also been generated in vitro identical to those seen in the patients described above. The cultures expand rapidly in the presence of primary leukemia and are almost exclusively Vδ1+ cells that express activation antigens CD25, HLA-DR, and CD69. These cells are also cytotoxic to primary ALL, lymphoid cell lines, and to K562 cells (FIG. 2) but are not cytotoxic to myeloid cell lines or third party AML. Donor cells cultured in the presence of AML or biphenotypic acute leukemia have also shown cytotoxicity to the primary leukemia suggesting that the effect may not be restricted to lymphoid leukemias. See Lamb, L. S., et al. Bone Marrow Transplantation 27: 601-606 (2001).

Allogeneic γδ T cells that closely resemble the γδ+ T cells in the patients described above were expanded in culture. See Lamb, L. S., et al. Bone Marrow Transplantation 27: 601-606 (2001). CD4-CD8-Vδ1+ cells that express activation markers CD69, HLA-DR, and CD25 proliferate rapidly in the presence of primary ALL (FIG. 2A). Augmentation of cell proliferation can be achieved by culturing the cells in the presence of immobilized pan-δ or Vδ1. These cells bind the primary ALL (FIG. 2B) and show specific lysis to the ALL (FIG. 2C). In the 50 patients accrued to date on this protocol, pure pre-B or B cell ALL has always been shown to stimulate γδ+ T cell proliferation with subsequent positive binding and cytotoxicity assays. Neither biphenotypic (CD33+) ALL nor AML of any subtype has generated a sustainable γδ+ T cell response.

FIG. 2 shows a culture of allogeneic γδ+ T cells with primary acute lymphoblastic leukemia. In FIG. 2A the γδ+ T cells grow in clusters on the ALL. When these cells are removed and exposed to fresh primary ALL blasts, the Vδ1+ cells bind the ALL, as indicated by the arrow (FIG. 2B), and are cytotoxic to both the ALL and K562 cells (FIG. 2C). The cytotoxicity of the γδ+ T cells is enhanced after exposure to immobilized Vδ1 antibody. The control targets represent cells that are normally unresponsive to cytolysis by CTL.

The in vitro allogeneic effect of γδ+ T cells is minimal and they do not initiate graft-versus-host disease. See Hacker, G., et al. J. Immunol. 149: 3984 (1992), Freedman, M. S., et al. J. Neuroimmunology 74:143 (1997), Norton, J., et al. Bone Marrow Transplantation 7: 205 (1991), Viale, M., et al. Bone Marrow Transplantation 10: 249 (1992), and Tsuji, S., et al. Eur. J. Immunol. 26: 420 (1996). These data strongly suggest a distinct role for γδ+ T cells against ALL independent of an allogeneic effect, and the direct recognition of a protein surface antigen on ALL by the γδ+ T cell.

### Example 3

The present invention shows for the first time that γδ+ T cells recognize ALL via the T cell receptor (TCR). Immune activation-specific cDNA microarray analysis of Vδ1+ cells from patients and in vitro ALL/γδ+ T cell co-cultures show mRNAs that are commonly upregulated with T cell receptor stimulation. Moreover, these cells do not use CD28 or 4-1 BB as co-stimulatory antigens, although LFA-1 is strongly expressed. As a result, NKG2D is co-stimulatory, as it is commonly expressed on NK cells, γδ+ T cells, and some γδ+ CTL. Also, perforin and granzyme are upregulated in γδ+ T cells that respond to ALL and are likely responsible for the cytotoxic effect.

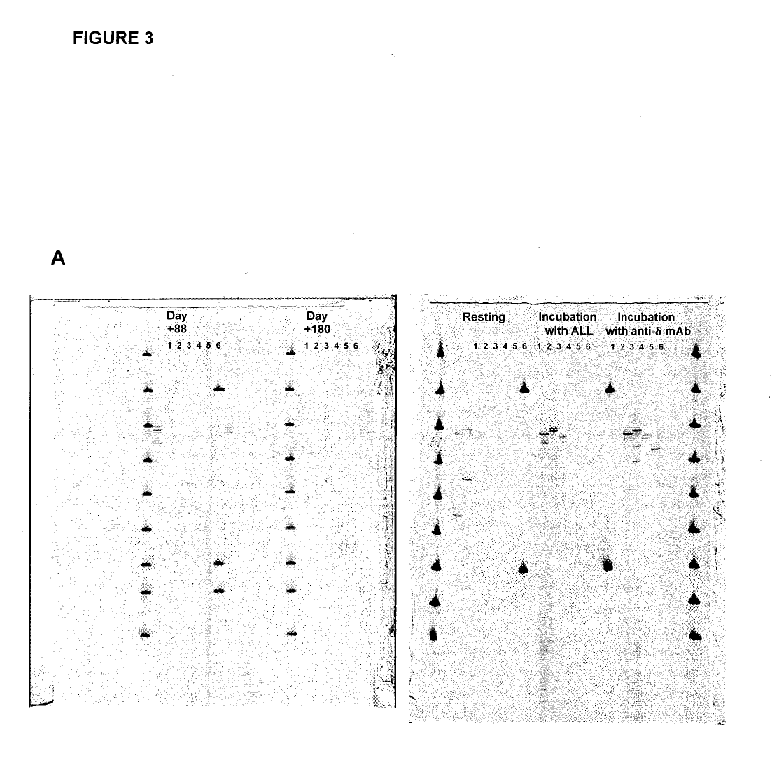

A high-resolution electrophoresis (spectratyping) experiment has shown that the number of Vδ1+ chains decrease and become more focused as the length of time γδ T cells are exposed to a leukemia that is stimulatory to the cell increases. This is illustrated in the gel photographs of high-resolution electrophoresis of Vδ chain cDNA presented in FIG. 3. Gel (A) is from a patient with increased γδ T cells showing almost exclusive Vδ1 transcripts with the loss of some transcripts over time. The same pattern appears in co-culture (B) of γδ-depleted PBMC with recipient ALL.

This “focusing” of the immune response suggests that the response may be clonal. In addition, preliminary sequencing data from patients and cultures where a Vδ1 response to primary leukemia has been documented shows a nearly identical sequence across patients and cultures. This shows the clonality of these cells, which in turn suggests a common stimulatory ligand (antigen).

### Example 4

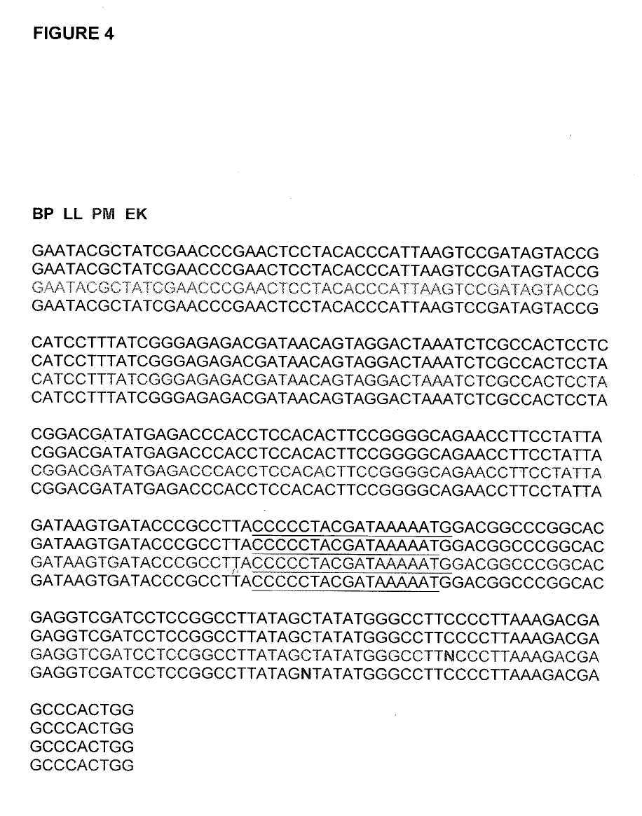

Sequencing analysis (SEQ ID NO.1) was performed on the Vδ1 chain from allogeneic BMT patients showing a γδ+ T cell response as well as cultures described above in which Vδ1+ cells were the predominant responders has been done (FIG. 4).

FIG. 4 shows the preliminary Vδ1 chain sequence from patients and controls. The underlined region represents an internal C region oligonucleotide probe.

At this time, the data indicate that a significant portion of at least one Vδ1 cDNA sequence is nearly identical for all responding cells. Thus, the response to leukemia is clonal.

### Example 5

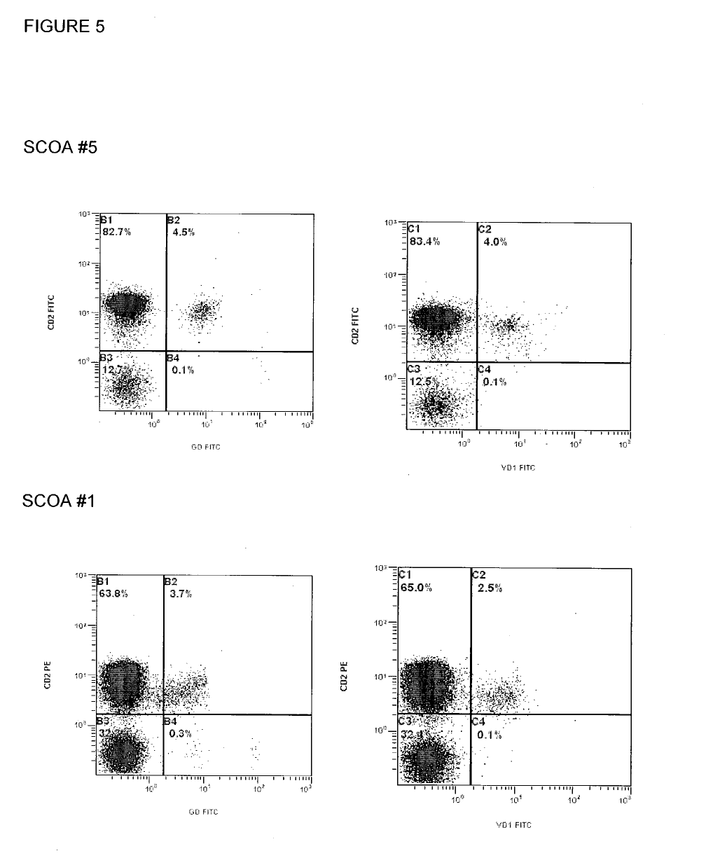

This experiment shows a small but specific Vδ1 response in two patients with AML M3 subtype (promyelocytic) as shown in FIG. 5. FIG. 5 is a flow cytometry graph indicating patients with acute promyelocytic leukemia, which demonstrate selection of a predominant Vδ1+ T cell population.

The first patient (SCOA #5) represents early relapsed AML (0.6% circulating blasts). Although the γδ population is within the expected range for a normal individual, the population is almost entirely Vδ1+. These results were similar to a previous patient that had demonstrated a vigorous in vitro Vδ1+ response to her autologous γδ+ T cell population. As shown in FIG. 5, this patient also shows a significant autologous Vδ1+ population at 1.5 years post-induction chemotherapy.

These findings are unique as the vast majority of both normal controls and leukemia patients are predominately Vδ2+ and, taken together with data from Duval et al. support an autologous Vδ1+ T cell response to selected leukemias. See Duval M., et al., Leukemia 9:863 (1995).

### Example 6

Based on the data described above, this experiment determined whether the specific immunogenicity of leukemia can lead to the isolation and purification of an ALL-associated antigen with potential as a therapeutic target.

The overall purpose of this experiment is to isolate and purify the ALL-associated antigen that is stimulatory to γδ+ T cells and is responsible for the in vitro and clinical findings detailed above. The experiment is similar to that used successfully by Olive et al. and involves TCR sequence analysis to document a clonal response of γδ+ T cells to RA to use a soluble γδ+ T cell receptor to isolate Listeria antigens that stimulate a γδ+ T cell response. See Olive, C., et al., Eur. J. Immunol. 22: 2587 (1992).

Sequence analysis of the TCR from Vδ1+ T cells isolated from patients and cultures that show a γδ+ T cell response to acute leukemia was used to determine if a similar TCR configuration is present across different patients and cultures. Since this response revealed a small number of responding T cell receptor sequences, a soluble TCR attached to a solid matrix was developed as a means of isolating a set of candidate antigens from leukemia cell lysates that bind to the recombinant TCR. Standard proteomics technology was used to identify candidate antigens.

Peripheral blood mononuclear cells (PBMC) and total RNA previously collected from post-BMT patients with increased Vδ1+ γδ T cells were used for sequence analysis of the γ and δ T cell receptor. For leukemia antigen binding studies, pediatric and adolescent patients presenting to the Children's Center for Cancer and Blood Disorders with an initial diagnosis of acute lymphoblastic leukemia (ALL) were entered consecutively following informed consent. An absolute requirement was that sufficient blasts can be obtained to permit the required assays. The attending pediatric oncologist determined the maximum amount of blood obtained from patients at any time, but at no time exceeded 50 ml. Patient accrual was approximately 20/year.

### Cell Preparation

Leukemia cells from patients were separated using density gradient centrifugation. Purity of the blast population was determined using flow cytometry with monoclonal antibodies (mAbs) specific for the patient's leukemia. Blasts were purified by either high-speed cell sorting or immunomagnetic selection using targeted mAbs. The final cell product was set aside for cryopreservation and storage for follow-up experiments.

### Polymerase Chain Reaction (PCR) Amplification of γδ T Cell Transcripts

Total RNA was isolated from peripheral blood mononuclear cells (PMMNC) using Trizol™ (Life Technologies, Rockville, Md.). First-strand cDNA was synthesized using the Qiagen one-step RT-PCR kit in 50 μL reactions containing the enzymes and buffer supplied by the manufacturer, 0.2 mM of each dNTP, 25 pmol of 5′ Vδ1 (5′-TCTGGATACAAGTGTGGC-3′) sense primer, 25 pmol of 3′ constant region gene antisense primer Cδ2 (5′-TTCACCAGACAAGCGACA-3′) and 2.5 U Hot Start™ Taq DNA polymerase (Qiagen; Valencia, Calif.). Reaction mixtures were amplified for 35 cycles using a thermal cycler (Perkin-Elmer Instruments). Following an initial incubation of 95° C., PCR conditions for amplification of 6 chain cDNA were 95° C. for 1 min, 50° C. for 1 min and 72° C. for 1 min for each cycle. The specificity of each PCR product was confirmed by Southern Blot analysis and hybridization with an internal C-region oligonucleotide probe Cδ3 (5′-GATGGTTTGGTATGAGGCTGA-3′). The process was repeated for the dominant y receptor clone identified in previous studies.

### Cloning of PCR Products

The PCR-amplified δ and γ chain cDNA were separated on a 1.25% agarose gel and isolated from a band approximately 400-600 bp. The purified 6 chain cDNA was then mixed directly with the pDrive UA cloning vector and 5 μl ligation master mix supplied by the manufacturer and incubated for 60 min at 15° C. The mixture was then transformed into Qiagen EZ™ Competent Cells. Ligation-reaction mixture (1-2 ml) was added to the competent cells, mixed, and incubated on ice for 5 min. Tubes were then incubated in a 42° C. water bath or heating block for 30 s and then on ice for 2 min. SOC medium (250 μl per tube) was added and 100 μl each transformation mixture was plated onto LB agar plates containing ampicillin. Colonies were transferred onto N⁺ nylon membrane discs and screened with full-length gel-purified Vδ and Vγ-specific probes to determine the total number of V61 chain cDNA clones, including those with CDNA cloned in the forward or reverse orientation. Positive clones in the forward orientation were selected and purified using conventional methods. These clones were further characterized in the reconstituted soluble T cell receptor system described below.

### Automated DNA Sequencing

Single-stranded DNA was prepared from positive cDNA clones using a standard method and sequenced by using an automated DNA sequencing system (Applied Biosystems; Foster City, Calif.). Cycle sequencing was performed by incubation of the single-stranded template (50-100 ng) with 8.0 μl dRhodamine dye terminator ready reaction mix (ABI) and 3.0 pmol dye primer in a total volume of 20 μL at 96° C. for 10 sec, 50° C. for 5 sec and 60° C. for 4 min×25 cycles. Extension products were purified by ethanol precipitation or by spin column. The pellet was resuspended in 5 μL deionized formamide, 1 μL 50 mM EDTA (pH 8.0), heated for 2 min at 90° C. and electrophoresed on a 6% polyacrylamide gel, using an ABI Prism™ 377 automated DNA sequencer. The ABI Sequence Analysis v. 3.0 software was then used to perform the sequence analysis.

### Construction of the Soluble T Cell Receptor

Baculovirus constructs containing the relevant y and 6 genes were produced in each case by PCR-amplifying TCR gene cDNAs derived from the patient material discussed above. For each, primers were designed that would truncate the genes just before the transmembrane regions, by insertion of termination codons. The cysteine codon for each chain that forms the interchain disulfide bond of the TCR was preserved in each case, such that the TCR sequence ends directly after the cysteine codon for Cδ and two codons below it for Cγ.

In addition, just prior to the termination codon, the Cδ genes include a 15 codon sequence whose product is recognized with high affinity by the E. coli enzyme Bir A [a BSP sequence, which can then be used to add a biotin to the C-terminus of the sTCR, if desired]. Each cDNA was cloned, sequence-verified, transferred into the baculovirus vector, and transfected into insect cells with baculovirus helper DNA to generate soluble TCR molecules.

Including a specific biotinylation site in each TCR allows for the possibility to easily make each STCR tetrameric, by complexing them with avidin (which has 4 high-affinity biotin sites). A virus encoding the sTCR was then generated by co-transfection of Sf9 moth cells with a mixture of commercially prepared BaculoGold “helper virus” DNA (Pharmingen) and the TCR-γδ vector-construct.

Affinity columns for purifying the recombinant soluble γδ TCRs were made by coupling TCRδ1, an anti-Cδ mAb, onto cyanogen activated sepharose beads. For large protein preps about 3 liters of insect cell culture supernatant was passed over a 3 ml affinity column at a time. To obtain cleaner preparations, it is possible to pre-pass the culture supernatant over an uncoupled column, a method that has been used successfully for purifying mouse γδ TCRs. The purified TCRs were then eluted with 50 mM diethylamine, neutralized, then dialyzed into PBS and concentrated.

This two-step purification technique yields TCR proteins that already show a fairly high degree of purity, and which retain activity in assays requiring native conformation for the TCR (see FIG. 6), which is necessary for ligand purification.

FIG. 6(A), is a SDS-PAGE analysis, stained with Coomassie Brilliant Blue, that shows mouse soluble TCRs purified as described; 4 ug/lane were loaded. 1:6.3, 5:1, and 6:1 denote the Vδ1/Vδ6.3, Vδ5/Vδ1, and Vδ6/Vδ1 TCRs, respectively; a γδ control TCR is also shown. Sizes of marked bands before and after reduction match those predicted for each of the TCRs based on length and N-glycosylation sites.

FIG. 6(B) shows purified soluble TCRs that bind to specific anti-TCR mAbs requiring native structure, in a competition assay. Appropriate anti-TCR mAbs were incubated for one hour either alone or after mixing with the indicated soluble TCR. The mAb and mAb/soluble TCR mixture were then compared for their ability to stain a T cell hybridoma cell bearing a TCR that is recognized by the mAb. Bound mAbs were detected with a FITC-labeled anti-rat or anti-hamster secondary antibody, as appropriate. As can be seen, the soluble TCRs effectively absorbed out mAbs, but only when in native form, since denatured (boiled) soluble TCRs were ineffective. None of the soluble TCRs had any effect on the binding of mAbs that were not specific for that particular TCR (not shown).

A Vδ1+ soluble TCR was then generated in this way, along with a control Vγ9/V≢2 soluble TCR. This control was used both to verify that any ligand that is purified binds specifically to the Vδ1+ TCR, but fails to bind to the Vγ9/Vδ2+ TCR, and to eliminate any candidates that may bind nonspecifically to γδ TCRs. The soluble Vδ1+ TCR was coupled to sepharose beads to generate an affinity column, or tetramerized and used in immunoprecipitation, to isolate natural ligand(s) present in whole cell lysates or cell membrane fractions from ALL tumor cells. The control Vγ9/Vδ2+ TCR was used to pre-clear the lysates and/or fractions, and to verify the specificity of any purified products.

### Proteomics

Proteins captured by the soluble T cell receptor were eluted and characterized using 2D gels (isoelectric focusing and SDS-PAGE) using several different pH gradients. The resulting gels were digitized and compared using Phoretix 2D software. Individual differences between gels were identified, and the differentially expressed proteins were analyzed using in-gel protein digestion coupled with peptide mass fingerprinting using tandem mass spectrometry obtained on an electrospray ionization quadruple time-of-flight mass spectrometer (Q-TOF spectrometry). See Borchers et al., Anal. Chem. 72: 1163-8 (2000). The data obtained by Q-TOF spectrometry was compared with known protein sequences. If the identified protein was novel, predicted cDNA sequences were compared with DNA databases as well as data recently made available through the human genome project.

### Statistics

This protocol relies on published preclinical work and clinical observations that have undergone exhaustive statistical analysis prior to peer review and publication. Data from protein microarrays was exported into the manufacturer's bioinformatics database and analyzed using their established protocols for protein expression. Other proteomics procedures are primarily biological assays to which population/biometric statistics do not yet apply, as this application is designed to provide preliminary data for a more widespread and focused study eventually culminating in a clinical trial.

### Evaluation Criteria

As stated earlier, the overall purpose of this experiment is to isolate and purify an ALL-associated antigen stimulatory to γδ+ T cells. The laboratory procedures that have been chosen for this invention have been successfully used in other settings to identify γδ+ T cell stimulatory antigens. If the sequences determined from analysis of patients and cultures that have shown an oligoclonal response to leukemia are restricted to a few clones, then the experiment is continued with the development of a soluble TCR as discussed above.

If multiple responding clones are seen, it may be necessary to develop multiple receptors with similar structure and then employ a more global proteomics approach focusing on membrane proteins.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties.

As various changes could be made in the above methods and compositions without departing from the scope of the invention, it is intended that all matter contained in the above description shall be interpreted as illustrative and not in a limiting sense.

What is claimed is:

1. A method for diagnosing a neoplasia disorder in a mammal, wherein said neoplasia disorder produces a ALL-associated cell surface antigen comprising:
a) providing a sample of biological material from said mammal;
b) contacting said biological material with antibodies specific for the antigen;
c) detecting the presence or absence of an immunological reaction product between said antibodies and said ALL-associated cell surface antigen, the presence of an immunological reaction product being indicative of said neoplasia disorder in said mammal.

2. The method according to claim 1, wherein said antibody is a monoclonal antibody.

3. The method according to claim 1, wherein said antibody specifically binds the same ALL-associated cell surface antigen as the monoclonal antibody produced by the hybridoma cell line having ATCC Accession No. #.

4. The method according to claim 1, wherein said ALL-associated cell surface antigen is specifically recognized by a γδ+ T cell receptor.

5. The method according to claim 1, wherein said γδ+ T cell receptor comprises a polynucleotide sequence having at least 70% sequence identity to SEQ ID NO.1.

6. The method according to claim 1, wherein said γδ+ T cell receptor comprises the polynucleotide sequence of SEQ ID NO.1.

7. The method according to claim 1, wherein said neoplasia disorder is a leukemia disorder.

8. The method according to claim 1, wherein said neoplasia disorder is selected from the group consisting of acute myelogenous leukemia, myeloblastic leukemia, promyelocytic leukemia, myelomonocytic leukemia, monocytic leukemia, erythroleukemia, megakaryoblastic leukemia, chronic myelogenous leukemia, acute lymphocytic leukemia, chronic lymphocytic leukemia, and hairy cell leukemia.

9. The method according to claim 1, wherein the biological material is selected from the group consisting of plasma, serum, cytosol fluid, ascites or tissue.

10. The method according to claim 1, wherein the neoplasia disorder is a leukemia disorder.

11. The method according to claim 1, wherein said neoplasia disorder is selected from the group consisting of acute myelogenous leukemia, myeloblastic leukemia, promyelocytic leukemia, myelomonocytic leukemia, monocytic leukemia, erythroleukemia, megakaryoblastic leukemia, chronic myelogenous leukemia, acute lymphocytic leukemia, chronic lymphocytic leukemia, and hairy cell leukemia.

12. The method of claim 1 wherein the antibody is a labeled antibody.

13. The method of claim 1 wherein the label is selected from the group consisting of an enzyme label, a radioisotope label and a fluorescent label.

14. The method of claim 1, wherein said immunological reaction is detected by an assay selected from the group consisting of Western blot assay, dot blot assay, ELISA sandwich assay, radioimmunoassay and flow cytometry assay.

15. An isolated antibody which specifically binds to an epitope of an ALL-associated cell surface antigen.

16. The antibody according to claim 14, wherein said antigen is capable of being recognized by a γδ+ T cell receptor having a partial polynucleotide sequence comprising SEQ ID NO: 1.

17. The antibody according to claim 14, wherein said antibody is a monoclonal antibody.

18. The antibody according to claim 14, wherein said antibody specifically binds to the same ALL-associated cell surface antigen as the monoclonal antibody produced by the hybridoma cell line having ATCC Accession No. #.

19. A method of treating a neoplasia disorder or a neoplasia-related disorder comprising administering to a subject in need of such treatment an effective amount of an antibody that specifically binds to an ALL-associated cell surface antigen.

20. The method according to claim 18, wherein said antibody has a cytotoxic activity.

21. The method according to claim 19, wherein said antibody has a cytotoxic activity that is selected from the group consisting of perforin, granzyme A, granzyme B and Fas-mediated.

22. The method according to claim 18, wherein said antibody is a monoclonal antibody.

23. The method according to claim 18, wherein said antibody specifically binds the same ALL-associated cell surface antigen as the monoclonal antibody produced by the hybridoma cell line having ATCC Accession No. #.

24. The method according to claim 18, wherein said ALL-associated cell surface antigen is specifically recognized by a γδ+ T cell receptor.

25. The method according to claim 18, wherein said γδ+ T cell receptor comprises a polynucleotide sequence having at least 70% sequence identity to SEQ ID NO.1.

26. The method according to claim 18, wherein said γδ+ T cell receptor comprises the polynucleotide sequence of SEQ ID NO.1.

27. The method according to claim 18, wherein said neoplasia disorder is a leukemia disorder.

28. The method according to claim 18, wherein said neoplasia disorder is selected from the group consisting of acute myelogenous leukemia, myeloblastic leukemia, promyelocytic leukemia, myelomonocytic leukemia, monocytic leukemia, erythroleukemia, megakaryoblastic leukemia, chronic myelogenous leukemia, acute lymphocytic leukemia, chronic lymphocytic leukemia, and hairy cell leukemia.

29. The method of claim 18, wherein said antigen is administered at a concentration of between 1.0 μg/kg and 10 mg/kg based on the body weight of the subject.

30. The method of claim 18, wherein said antigen is administered at a concentration of between 0.1 and 2 mg/kg, based on the body weight of the subject.

31. The method of claim 18, wherein said antigen is coupled or conjugated with a carrier.

32. An isolated polynucleotide which encodes for an acute lymphoblastic leukemia specific γδ+ T cell receptor.

33. The isolated polynucleotide according to claim 32, wherein said polynucleotide has at least 70% sequence identity with SEQ ID NO. 1.

34. The isolated polynucleotide according to SEQ ID NO. 1.

35. A method of preventing and treating a neoplasia disorder in a subject that is in need of such prevention and treatment comprising administering to the subject an activated γδ+ T cell-rich composition in combination with one or more conventional cancer treatment agents.

36. The method according to claim 35, wherein said neoplasia disorder is a leukemia disorder.

37. The method according to claim 35, wherein said neoplasia disorder is selected from the group consisting of acute myelogenous leukemia, myeloblastic leukemia, promyelocytic leukemia, myelomonocytic leukemia, monocytic leukemia, erythroleukemia, megakaryoblastic leukemia, chronic myelogenous leukemia, acute lymphocytic leukemia, chronic lymphocytic leukemia, and hairy cell leukemia.

38. The method according to claim 35, wherein said conventional cancer treatment agent is selected from the group consisting of radiation therapy, chemotherapy, immunotherapy, surgical therapy and cryotherapy.

39. A method of preventing and treating neoplasia in a subject that is in need of such prevention and treatment comprising administering to the subject an antibody that is specific for an ALL-associated cell surface antigen in combination with one or more conventional cancer treatment agents.

40. The method according to claim 39, wherein said ALL-associated cell surface antigen is specifically recognized by a γδ+ T cell receptor.

41. The method according to claim 39, wherein said γδ+ T cell receptor comprises a polynucleotide sequence having at least 70% sequence identity to SEQ ID NO.1.

42. The method according to claim 39, wherein said γδ+ T cell receptor comprises the polynucleotide sequence of SEQ ID NO.1.

43. The method according to claim 39, wherein said conventional cancer treatment agent is selected from the group consisting of radiation therapy, chemotherapy, immunotherapy, surgical therapy and cryotherapy.

44. The method according to claim 39, wherein said neoplasia disorder is a leukemia disorder.

45. The method according to claim 39, wherein said neoplasia disorder is selected from the group consisting of acute myelogenous leukemia, myeloblastic leukemia, promyelocytic leukemia, myelomonocytic leukemia, monocytic leukemia, erythroleukemia, megakaryoblastic leukemia, chronic myelogenous leukemia, acute lymphocytic leukemia, chronic lymphocytic leukemia, and hairy cell leukemia.

46. A pharmaceutical composition comprising activated γδ+ T cell-rich composition in combination with one or more conventional cancer treatment agents, and a pharmaceutically acceptable carrier.

47. A pharmaceutical composition comprising an antibody that is specific for an ALL-associated cell surface antigen in combination with one or more conventional cancer treatment agents, and a pharmaceutically acceptable carrier.

48. A pharmaceutical composition comprising an isolated antibody which specifically binds to an epitope of an ALL-associated cell surface antigen, and a pharmaceutically acceptable carrier.

49. The pharmaceutical composition according to claim 46, wherein said antigen is capable of being recognized by a γδ+ T cell receptor having a partial polynucleotide sequence comprising SEQ ID NO: 1.

50. The pharmaceutical composition according to claim 46, wherein said antibody is a monoclonal antibody.

51. The pharmaceutical composition according to claim 46, wherein said antibody specifically binds to the same ALL-associated cell surface antigen as the monoclonal antibody produced by the hybridoma cell line having ATCC Accession No. #.
